SEQUENCE LISTING (38 sequences)

SEQ ID NO: 1 - protein, 20 aa, synthetic construct
  REGION 1..20: note = Synthetic construct
DMATEMVKYSNANILSQAGQ

SEQ ID NO: 2 - protein, 17 aa, synthetic construct
  REGION 1..17: note = Synthetic construct
ISQAVHAAHAEINEAGR

SEQ ID NO: 3 - protein, 14 aa, synthetic construct
  REGION 1..14: note = Synthetic construct
EAWGALANWAVDSA

SEQ ID NO: 4 - protein, 162 aa, synthetic construct
  REGION 1..162: note = Synthetic construct
MRGSHHHHHHGSMRKQIRGLTQASTNAEDGISSVQTAEGALTEVHDMLQRMNELAIQAANGTDMATEMVKYSNANILSQAGQDMATEMVKYSNANILSQAGQDMATEMVKYSNANILSQAGQISQAVHAAHAEINEAGREAWGALANWAVDSARGSHHHHHH

SEQ ID NO: 5 - protein, 471 aa, synthetic construct
  REGION 1..471: note = Synthetic construct
MVVQHNMQAMNANRMLNVTTLTEVHSMLQRMNELAVQASNGMVVQHNMTAANANRMGETHSILQRMNELATQAANMVVQHNLTAMNANRQLVGTTGMVVQHNMQAANANRMLGITSVHSMLQRMNELAVQAASNGTNSMVVQHNMQAANANRMLNVTTLTEVHSMLQRMNELATQSANGLTEVHSMLQRMNELAVQSSNGDMAEEMVEYSKNNILAQAGQSMLAQANQSMAEEMVNYSKNNILAAQAGQSMLAQANQMAKEMVNYSKNNILAQAGQSMLAQANDMAEEMVTYSKNNILAQAGQSMLAQANQMVVQHNLRAMNSNRMLGITQSAQRSLLGAVQNRLEHTINNNEAHSILQRMNELAVQGANDVEYSKNNILAQAGQSMLAQANQMVVQHNLRAMNSNRMLSITQDMATEMVKFSNSNILAQAGQMVVQHNLRAMNANRMLGITTEVHDMLQRMNELAVKAAN

SEQ ID NO: 6 - protein, 20 aa, synthetic construct
  REGION 1..20: note = Synthetic construct
MVVQHNMQAMNANRMLNVTT

SEQ ID NO: 7 - protein, 21 aa, synthetic construct
  REGION 1..21: note = Synthetic construct
LTEVHSMLQRMNELAVQASNG

SEQ ID NO: 8 - protein, 15 aa, synthetic construct
  REGION 1..15: note = Synthetic construct
MVVQHNMTAANANRM

SEQ ID NO: 9 - protein, 19 aa, synthetic construct
  REGION 1..19: note = Synthetic construct
GETHSILQRMNELATQAAN

SEQ ID NO: 10 - protein, 21 aa, synthetic construct
  REGION 1..21: note = Synthetic construct
MVVQHNLTAMNANRQLVGTTG

SEQ ID NO: 11 - protein, 20 aa, synthetic construct
  REGION 1..20: note = Synthetic construct
MVVQHNMQAANANRMLNVTT

SEQ ID NO: 12 - protein, 21 aa, synthetic construct
  REGION 1..21: note = Synthetic construct
LTEVHSMLQRMNELATQSANG

SEQ ID NO: 13 - protein, 21 aa, synthetic construct
  REGION 1..21: note = Synthetic construct
LTEVHSMLQRMNELAVQSSNG

SEQ ID NO: 14 - protein, 29 aa, synthetic construct
  REGION 1..29: note = Synthetic construct
DMAEEMVEYSKNNILAQAGQSMLAQANQS

SEQ ID NO: 15 - protein, 28 aa, synthetic construct
  REGION 1..28: note = Synthetic construct
MAEEMVNYSKNNILAAQAGQSMLAQANQ

SEQ ID NO: 16 - protein, 26 aa, synthetic construct
  REGION 1..26: note = synthetic construct
MAKEMVNYSKNNILAQAGQSMLAQAN

SEQ ID NO: 17 - protein, 28 aa, synthetic construct
  REGION 1..28: note = synthetic construct
DMAEEMVTYSKNNILAQAGQSMLAQANQ

SEQ ID NO: 18 - protein, 20 aa, synthetic construct
  REGION 1..20: note = Synthetic construct
MVVQHNLRAMNSNRMLGITQ

SEQ ID NO: 19 - protein, 20 aa, synthetic construct
  REGION 1..20: note = Synthetic construct
SAQRSLLGAVQNRLEHTINN

SEQ ID NO: 20 - protein, 20 aa, synthetic construct
  REGION 1..20: note = Synthetic construct
NEAHSILQRMNELAVQGAND

SEQ ID NO: 21 - protein, 21 aa, synthetic construct
  REGION 1..21: note = Synthetic construct
VEYSKNNILAQAGQMLAQANQ

SEQ ID NO: 22 - protein, 20 aa, synthetic construct
  REGION 1..20: note = Synthetic construct
MVVQHNLRAMNSNRMLSITQ

SEQ ID NO: 23 - protein, 20 aa, synthetic construct
  REGION 1..20: note = Synthetic construct
DMATEMVKFSNSNILAQAGQ

SEQ ID NO: 24 - protein, 19 aa, synthetic construct
  REGION 1..19: note = Synthetic construct
MVVQHNLRAMNANRMLGIT

SEQ ID NO: 25 - protein, 19 aa, synthetic construct
  REGION 1..19: note = Synthetic construct
TEVHDMLQRMNELAVKAAN

SEQ ID NO: 26 - protein, 20 aa, synthetic construct
  REGION 1..20: note = Synthetic construct
MKVKVLSLLVPALLVAGAAN

SEQ ID NO: 27 - protein, 20 aa, synthetic construct
  REGION 1..20: note = Synthetic construct
VDVGATYYFNKNMSTYVDYK

SEQ ID NO: 28 - protein, 36 aa, synthetic construct
  REGION 1..36: note = Synthetic construct
MRGSHHHHHHGMASMTGGQQMGRDLYDDDDKDHPFT

SEQ ID NO: 29 - protein, 133 aa, synthetic construct
  REGION 1..133: note = Synthetic construct
APTSSSTKKTQLQLEHLLLDLQMILNGINNYKNPKLTRMLTFKFYMPKKATELKHLQCLEEELKPLEEALNLAPSKNFHIRPRDLISDINVIVLELKGSETTFMCEYADETATIVEFLNRWITFSQSIISTLT

SEQ ID NO: 30 - protein, 133 aa, synthetic construct
  REGION 1..133: note = Synthetic construct
APTSSSTKKTQLQLEHLLLDLQMILNGINNYKNPKLTRMLTFKFYMPKKATELKHLQCLEEELKPLEEALNLAPSKNFHLRPRDLISDINVIVLELKGSETTFMCEYADETATIVEFINRWITFSQSIISTLT

SEQ ID NO: 31 - protein, 8 aa, synthetic construct
  REGION 1..8: note = Synthetic construct
DYKDDDDK

SEQ ID NO: 32 - protein, 6 aa, synthetic construct
  REGION 1..6: note = Synthetic construct
HHHHHH

SEQ ID NO: 33 - protein, 9 aa, synthetic construct
  REGION 1..9: note = Synthetic construct
YPYDVPDYA

SEQ ID NO: 34 - protein, 17 aa, synthetic construct
  REGION 1..17: note = Synthetic construct
MVVQHNLQAMNSNRMLG

SEQ ID NO: 35 - protein, 16 aa, synthetic construct
  REGION 1..16: note = Synthetic construct
AGAIKKVSTQRSALGA

SEQ ID NO: 36 - protein, 19 aa, synthetic construct
  REGION 1..19: note = Synthetic construct
MATEMVKYSNANILSQAGQ

SEQ ID NO: 37 - protein, 20 aa, synthetic construct
  REGION 1..20: note = Synthetic construct
MVVQHNMQAANANRMLGITS

SEQ ID NO: 38 - protein, 22 aa, synthetic construct
  REGION 1..22: note = Synthetic construct
VHSMLQRMNELAVQAASNGTNS

(19) United States
(12) Patent Application Publication
(10) Pub. No.: US 2025/0375501 A1
(43) Pub. Date: Dec. 11, 2025

(54) IMMUNOTHERAPY FOR THE TREATMENT AND PREVENTION OF INFLAMMATORY BOWEL DISEASE

(71) Applicant: THE UAB RESEARCH FOUNDATION, Birmingham, AL (US)

(72) Inventors: Charles O. Elson, Birmingham, AL (US); Wayne Duck, Pell City, AL (US); Qing Zhao, Birmingham, AL (US); Katie Alexander, Homewood, AL (US)

(73) Assignee: THE UAB RESEARCH FOUNDATION, Birmingham, AL (US)

(21) Appl. No.: 19/225,127

(22) Filed: Jun. 2, 2025

Related U.S. Application Data
(62) Division of application No. 17/426,234, filed on Jul. 28, 2021, now Pat. No. 12,350,317.
(60) Provisional application No. 62/802,873, filed on Feb. 8, 2019.

(51) Int. Cl. A61K 38/20; A61K 31/436; A61K 38/16; A61K 45/06; A61P 29/00

(52) U.S. Cl. CPC A61K 38/2013; A61K 31/436; A61K 38/164; A61K 45/06; A61P 29/00

(57) ABSTRACT

Provided herein are methods and compositions for treating and preventing inflammatory bowel disease.

### PRIOR RELATED APPLICATION

This application is a divisional of U.S. patent application Ser. No. 17/426,234 filed on Jul. 28, 2021, which is a U.S. national stage application under 35 USC § 371 of PCT/US2020/017319 filed on Feb. 7, 2020, which claims the benefit of U.S. Provisional Application No. 62/802,873 filed on Feb. 8, 2019, which is hereby incorporated by reference in its entirety.

### STATEMENT REGARDING FEDERALLY FUNDED RESEARCH

This invention was made with government support under Grant No. DK071176 awarded by the National Institutes of Health. The government has certain rights in the invention.

### SEQUENCE LISTING

The instant application contains a Sequence Listing in XML format. The Sequence Listing, named UAB-191US2_ST26_SEQ_LIST.xml, which was created on May 19, 2025, is 49 KB in size, and is hereby incorporated by reference in its entirety.

### BACKGROUND

As a global disease, the prevalence of inflammatory bowel disease (IBD) is over 0.3% in developed countries and continues to rise in developing countries. Its major forms, Crohn's disease and ulcerative colitis, are associated with substantial morbidity and huge medical care costs. Current treatments and therapies typically treat the symptoms of the disorders and do not provide curative treatment.

### SUMMARY

Provided herein are methods for treating or preventing inflammatory bowel disease. The methods comprise administering to a subject having inflammatory bowel disease or at risk of developing inflammatory bowel disease (a) an effective amount of a polypeptide comprising one or more flagellin T-cell receptor (TCR) epitopes; and (b) an effective amount of an agent that reduces flagellin antigen-specific memory T cells and/or increases regulatory T cells in the subject. In some embodiments, the agent that that reduces flagellin antigen-specific memory T cells and/or increases regulatory T cells in the subject is a metabolic inhibitor.

Also provided are methods for delaying or reducing the intensity of a relapse or flare of an inflammatory bowel disease in a subject. The methods comprise administering to a subject (a) an effective amount of a polypeptide comprising one or more flagellin T-cell receptor (TCR) epitopes; and (b) an effective amount of an agent that reduces flagellin antigen-specific memory T cells and/or increases regulatory T cells in the subject. In some embodiments, the agent that that reduces flagellin antigen-specific memory T cells and/or increases regulatory T cells in the subject is a metabolic inhibitor.

### BRIEF DESCRIPTION OF THE DRAWINGS

The present application includes the following figures. The figures are intended to illustrate certain embodiments and/or features of the compositions and methods, and to supplement any description(s) of the compositions and methods. The figures do not limit the scope of the compositions and methods, unless the written description expressly indicates that such is the case.

FIGS. 1A-1B show that CD4 memory T (T_{M}) cells are widely distributed in CBir1 T cell receptor transgenic (CBir1 TCR Tg) mice. This transgenic mouse line is specific for CBir1 flagellin.

FIG. 2 shows that a subset of Crohn's patients has broad IgG reactivity to microbiota flagellins.

FIG. 3 is a schematic of a mechanism for ablating CD4 T_{M} cells by administering a polypeptide comprising one or more flagellin TCR epitopes and a metabolic inhibitor to a subject. Inactivation of microbiota-flagellin specific T_{M} cells or inducing Treg cells via T cell receptor stimulation and inhibition of mTORC results in the ablation of microbiota-reactive T_{M} cells and an altered ratio of Treg/effector T (T_{E}) cells.

FIGS. 4A-4B show the structure and validation of a multi-epitope peptide (MEP1). (A) MEP1 is a 162 amino acid long peptide including 3 repeats of a CBir1Tg CD4⁺ T cell epitope and 1 repeat of OT-II CD4⁺ T cell epitope. (B) CBir1Tg CD4⁺ T cells were isolated from the spleen and labeled with (5(6)-Carboxyfluorescein N-hydroxysuccinimidyl ester (CFSE). After 90 hrs in culture with irradiated APCs isolated from C57BL/6 mice plus MEP1 or CBir1 p456-475, at indicated concentrations, CBir1Tg CD4⁺ T cell proliferation was determined with FACS analysis.

FIGS. 5A-5P show that rapamycin inhibits microbiota-specific CD4⁺ T cell activation and proliferation through metabolic targeting in vitro, and generates suppressive Treg cells. CBir1Tg CD4⁺CD44− naïve T cells were isolated from the spleen and co-cultured with irradiated antigen-presenting-cells (APCs) isolated from C57BL/6 mice, in the presence of 1 μg/ml of MEP1 with (blue) or without (red) 100 nM rapamycin. Phosphorylation of S6K in CBir1Tg CD4⁺ T cells was examined after 20 hrs of stimulation. CBir1Tg CD4⁺ T cell viability, uptake of fluorescent glucose analog 2-NBDG, proliferation, and Treg induction were determined with FACS after 90 hrs of co-culture. OVA p323-339 stimulation was used as a negative control (grey). Representative flow plots are shown in (A-D, and I), and statistics of 3 replicated experiments are shown in (E-H, and J). For in vitro and in vivo suppression assays, naïve CD4+ cells were isolated from CBir1Tg. Foxp3gfp spleen and cultured with MEP1 and rapamycin for 6 days for Treg induction. Then live CD4⁺CD25⁺GFP⁺ Treg cells were sorted with flow cytometry as CBir1Tg iTreg Rapa. Live CD4⁺CD25⁺GFP⁺ cells freshly isolated from naïve CBir1Tg.Foxp3gfp spleen were used as control (CBir1Tg tTreg) for suppression assay in vitro. CFSE labeled responder CD4+CD44− (Tn) cells were isolated from congenic CBir1Tg or C57BL/6 mouse and co-cultured with indicated ratios of CBir1Tg Treg cells in the presence of 1 μg/ml MEP1 or anti-mouse-CD3 and APCs for 3.5 days. Representative graphs of antigen-specific and bystander suppression are shown in (K and M), respectively, and accumulated percentage of suppression are shown in (L and N). For in vivo suppression, CFSE labeled responder Tn cells (CBir1Tg or OT-II Tg) were co-transferred with CBir1Tg iTreg Rapa cells or naïve CBir1Tg CD4+ cells (Ctrl) into congenic C57BL/6 recipients at 1:1 ratio. Recipient mice were then challenged i.p. with 30 μg CBir1 flagellin for antigen-specific suppression or 10 μg MEP1 for bystander suppression, and the proliferation of responder cells post 6 days of challenge are shown in (O and P), respectively.

FIGS. 6A-6G show that simultaneous rapamycin treatment with peripheral antigen activation prevents the development of naïve CD4+ T cell mediated colitis in immunocompromised mice. Strategy of adoptive transfer and colitis induction is shown in (A). Rag-/- mice were transferred with 1×10⁶ CD4+CD25− naïve T cells isolated from the spleen of CBir1Tg mice, followed by 5 μg of MEP i.v. injection on Day 1. Recipient mice were then treated with rapamycin (1 μg/g/day) or vehicle alone (0.2% CMC) on days 1-5 and days 8-12. Rag-/- mice receiving CBir1Tg CD4+CD25− cells without MEP1 stimulation served as colitic controls. Weight loss of recipient mice are shown in (B). Mice were sacrificed on day 18, when cecum and colon tissue were collected for histological analysis. Representative microscopic views of murine distal colon in each group are shown in (C), and histology severity scores are shown in (D). Colonic lamina propria cells were isolated from the recipient mice upon sacrifice for analyses of CD4⁺ T_{E} cell composition, and the absolute numbers of total CD4⁺, Th1 and Th17 of transferred CBir1Tg cells in each group are shown in (E-G).

FIGS. 7A-7E show that microbiota flagellin-specific CD4⁺ T_{M} cells are present locally in the intestine and circulating in the periphery. (A) TCRβδ-/- mice were transferred with 8×10⁵ naïve CD4⁺ T cells from CBir1Tg.CD45.1 mice and 2×10⁵ naïve CD4+ T cells from B6.CD45.2 mice, followed by subcutaneous injection of 3 μg MEP1 on the next day. Recipient mice were maintained under SPF environment for 6 months for microbiota flagellin-specific CD4+ T_{M} response development. Upon sacrifice, lymphocytes were isolated from the small bowel (SB), large bowel (LB), mLN, spleen, inguinal lymph nodes (pLN), and bone marrow (BM) of recipient mice and analyzed with FACS. (B) Absolute numbers of CBir1-specific (CD45.1+) CD4⁺CD44⁺CD127⁺ T_{M} cells in different tissues. (C) Percent of CCR7 expression on CD4+ T_{M} cells in different tissues, whereas the absolute numbers of CD4⁺ T_{M} cells expressing CCR7 is shown in (D). Flow sorted CD45.1⁺CD4⁺CD44⁺CD127⁺ T_{M} cells from the spleen of colitic recipient mice were used for the 2° transfer. For 2° transfer, Rag-/- mice received 5×10⁵ Tm or CBir1Tg CD4+CD44− (Tn) cells on Day 0, followed by i.p. injection of rapamycin (1 μg/g/day) resuspended in 0.2% CMC or vehicle alone on days 1-5 and days 8-12. Histological analysis on cecal and colonic tissues on day 20 is shown in (E).

FIGS. 8A-8M show that rapamycin prevents the development of CD4⁺ T_{M} while promotes Treg in vivo, in an antigen-specific manner. (A) C57BL/6.CD45.2 mice were adoptively transferred with 2×10⁶ CBir1Tg.CD45.1 CD4⁺CD44⁻ naïve splenic T cells (red arrow), followed by immunization with 50 μg CBir1 flagellin and lug cholera toxin (CT) on Day 0 and Day 7 (black arrow). Recipient mice without immunization were used as controls. Mice were sacrificed or challenged with CBir1 flagellin (which were then sacrificed 7 days post challenge) on indicated days post transfer for the enumeration of remaining donor CD4+T cells in the spleen (B). (C) Adoptive transfer, immunization and inhibition strategy with same color indications as in (A). Blue arrow indicates rapamycin i.p. injection at 1 μg/g body weight/day, while mice in the control group were treated only with drug vehicle 0.2% CMC. Day 28 post transfer, lymphocytes were isolated from the recipient mice and analyzed with FACS. Absolute numbers of CD4⁺CD45.1⁺ T cells in spleen, mLN, BM and intestine are shown in (D). Representative plots of donor CD4⁺ T_{M}, Treg, effector Th1 and Th17 cells (T_{E}), and Tfh in the spleen are shown in (E and H) (red: CMC group; blue: rapa group), whereas combined data of the percentage of donor CD4+ TM and ratios of Treg/T_{E} based on absolute numbers in the spleen and mLN are shown in (F and G). Serum IgG antibodies specific to CBir1 flagellin and CTB on Day28 are shown in (I and J). (K) shows adoptive transfer, immunization, and challenging strategy. Red dashed arrow indicates challenging with 3 μg MEP1 i.v. on day 28, and mice were then sacrificed 7 days after. Percentages of donor Treg, and ratios of donor Treg/T_{E} in the spleen are shown in (L and M).

FIGS. 9A-9C show that rapamycin has no effect on host CD4⁺ T_{M} and Treg development. B6.CD45.2 mice were transferred with 2×10⁶ naïve CD4⁺ T cells from CBir1Tg. CD45.1 mice, followed by i.p. immunization of CBir1 flagellin on day 0 and 7. Recipient mice were treated with 5 days of rapamycin injection at 1 μg/g body weight/day after each immunization, while the control group were treated only with drug vehicle 0.2% CMC. On day 28, lymphocytes were isolated from the spleen and mLN and analyzed with FACS. Percentages of CD4⁺ T_{M} cells in the recipient mice are shown in (A and B), whereas host CD4⁺ Treg cells and their ratio over effector Th1 and Th17 cells (TE) is shown in (C).

FIGS. 10A-10I show that the combination of rapamycin and metformin ablates pre-existing microbiota antigen-specific CD4⁺ T_{M} cells in the periphery in vitro and in vivo. (A) CBir1Tg CD4⁺CD44⁺ T_{M} cells were isolated from the spleen, labeled with CFSE, and co-cultured with irradiated APCs isolated from C57BL/6 mice in the presence of 1 μg/ml of MEP1, and 100 nM rapamycin, 1 mM metformin, or both. Absolute numbers of live and proliferating CD4⁺ T cells post 90 hrs of culturing are shown respectively. OVA p323-339 stimulation was used as negative control. (B) Strategy of adoptive transfer, immunization, and ablation of donor CD4⁺ T_{M} cells. C57BL/6.CD45.2 mice were adoptively transferred with 2×10⁶ CBir1Tg.CD45.1 CD4⁺CD44⁻ naïve splenic T cells on Day−1, followed by immunization with 50 μg CBir1 flagellin and 1 μg CT on Day 0 and Day 7 for CD4⁺ T_{M} induction. Recipient mice were challenged with 3 μg MEP1 i.v. on Day 28 and Day 35 (black dashed arrow), then followed by i.p. injection of rapamycin (1 μg/g/day), metformin (150 μg/g/day), or the combination of both for 5 days (grey arrow). Mice without challenge and mice treated with 0.2% CMC post challenge were used as controls. Lymphocytes were isolated from the spleen on Day 42 and analyzed with FACS. Representative plots of donor/host CD4⁺, donor CD4⁺ T_{M}, Treg, and Tfh are shown in (C), whereas statistics of corresponding absolute numbers are shown in (D-G). Serum IgG antibodies specific to CBir1 flagellin and CTB in the recipient mice are shown in (H and I), respectively.

FIGS. 11A-11G show that metabolic checkpoint inhibition dampens antigen-specific CD4⁺ T cell recall response post ablation. (A) Strategy of adoptive transfer, immunization, and ablation of donor CD4⁺ T_{M} cells. C57BL/6.CD45.2 mice were adoptively transferred with 2×10⁶ CBir1Tg.CD45.1 CD4⁺CD44⁻ naïve splenic T cells on Day−1, followed by immunization with 50 μg CBir1 flagellin and 1 μg CT on Day 0 and Day 7 for CD4⁺ T_{M} induction. Recipient mice were challenged with 3 μg MEP1 i.v. on Day 28 and Day 35 (black dashed arrow), then followed by i.p. injection of rapamycin (1 μg/g/day), metformin (150 μg/g/day), or the combination of both for 5 days (grey arrow). Mice without MEP1 challenge on Day 28 and 35 were used as no treatment controls; and mice treated with 0.2% CMC post MEP1 challenge were used as drug vehicle controls. All groups received a MEP1 challenge on Day 56. Lymphocytes were isolated from the spleen on Day 63 and analyzed with FACS. Representative plots of donor CD4⁺ T_{M} are shown in (B), whereas statistics of absolute numbers of donor CD4⁺ T cells, T_{EM} cells, T_{CM} cells, and CD27 expression in donor CD4⁺ T cells were shown in (C-F). RNA-sequencing was performed with flow sorted donor CD4⁺ T cells (n=3-4 mice per group), and log2 fold change of gene expression levels in treated mice compared to no treatment group were shown in (G).

FIGS. 12A-12M show that metabolic checkpoint inhibition dampens the survival and proliferation of circulating microbiota antigen-specific CD4⁺ T cells isolated from Crohn's patients. (A) At least 10⁷ PBMCs isolated from Crohn's patients with high serologic responses to multiple Lachnospiraceae flagellins (CD high, n=10), Crohn's patients with low serologic responses to Lachnospiraceae flagellins (CD low, n=10), and healthy controls (HC, n=10) were stimulated with a mix of four flagellin antigens (Fla mix, composed of A4 Fla-3, A4 Fla-4, 14-2 Fla-1 and CBir1 Fla, 10 ug/ml each) and 1 μg/ml anti-CD28 and anti-CD40 for 7 hours before enrichment with CD154-biotin/anti-biotin magnetic beads. Cells stimulated without antigens, but only anti-CD28/CD40 were used to assess non-specific CD154 up-regulation. Cells were then stained for CD154, CD69, CD45RO, and intracellular cytokines, and analyzed by flow cytometry. Representative plots of CD69⁺CD154⁺ cells in CD3⁺CD4⁺ gate pre- and post-enrichment are shown in (B), and percent of CD154⁺ cells in total viable CD4⁺ cells are shown in (C), where each dot represents an individual. Representative flow plots and statistics of IFNγ expression in CD4⁺CD154⁺ cells post stimulation are shown in (D and E). A representative flow plot and the percentage of CD45RO⁺ cells in CD69⁺CD154⁺ cells post-enrichment and in CD3⁺CD4⁺ cells pre-enrichment is shown in (F and G). Microbiota flagellin-specific CD4⁺ T cells were labeled with proliferation dye and re-stimulated with autologous APCs plus Fla mix in the presence of 50 nM rapamycin, 1 mM metformin, or both. Phosphorylation of S6K was examined after 6 hours of stimulation (H and K), whereas CD4⁺ T cell survival (I and L) and proliferation (J and M) were examined after 6-7 days.

FIG. 13 shows that rapamycin has no effect on microbiota flagellin-specific CD4⁺ T cell cytokine production. Expanded CD154⁺ antigen-specific CD4⁺ T cells were re-stimulated with autologous APCs and Fla mix, Fla mix with rapamycin, Fla mix with metformin, or Fla mix with rapamycin and metformin for 6-7 hours, with BFA added for the last 4 hours. CD4⁺ T cells stimulated with CD3/CD28 beads were used as a positive control, whereas cells with no antigen added were used as a negative control. The cells were then stained for cytokines (TNF and IL-17A), activation of mTOR via the downstream marker phospho-S6K and analyzed by flow cytometry.

FIG. 14 shows a Crohn's patient/s CD4+ T cell response to a flagellin multiepitope peptide (MEP1, also referred to as MEPZ) compared to a mixture of the recombinant flagellin proteins. The multiepitope peptide stimulated both Tconv (CD154+) and Treg (CD137+) CD4+ T cells that are reactive to microbiota flagellin. Numbers in the upper right hand corner of each panel show absolute numbers of cells that are reactive to different stimulations.

### DETAILED DESCRIPTION

Currently there is no cure for Crohn's disease or ulcerative colitis, the two main types of IBD. IBD is a chronic condition, and people with IBD will typically need treatment throughout their lives. As shown herein, the pathogenesis of IBD is due to an abnormal immune response, particularly a CD4⁺ T cell response, to microbiota antigens in genetically-susceptible hosts. Microbiota flagellins, especially those that belong to the Lachnospiraceae family, were identified as immunodominant antigens driving the adaptive cellular and humoral immune responses in murine colitis models. Similarly, over half of the patients with Crohn's disease have elevated serological reactivity to CBir1 and its related flagellins, which is generally companied with a more complicated clinical course.

Much has been learned by focusing on CD4+T effector (T_{E}) cells (Th1, Th17, and Th1/Th17), but less is known about CD4+T memory (T_{M}) cells reactive to the gut microbiota. Long-lived microbiota-specific CD4+T_{M} cells are present in healthy human individuals, and can be generated during intestinal inflammation and infections, as shown in murine models. At steady state, these CD4+T_{M} cells are widely distributed in tissues including the colonic lamina propria (LP), mesenteric lymph nodes (mLNs), spleen, blood, and bone marrow (BM). The fact that they responded quickly by drastically proliferating and producing cytokines such as IFNγ and IL-17A when challenged with a microbiota antigen in the periphery in mice and gave rise to secondary colitis indicates that microbiota-specific CD4⁺ T_{M} cells serve as a potential pathogenic CD4+ T effector cell reservoir for later-on intestinal inflammations.

Resting CD4+ T naïve (T_{N}) and T_{M} cells keep a low level of metabolism but undergo a profound metabolic transition from using mitochondrial oxidative phosphorylation (OXPHOS) and fatty acids oxidation to predominantly engaging glycolysis when stimulated through the T cell receptor (TCR) and co-stimulatory molecules. This metabolic status switch is primarily controlled by the mammalian target of rapamycin (mTOR) complex. Thus, activation of mTOR is needed for T cell expansion and is an inescapable metabolic checkpoint for activating T_{N} and T_{M} cells. In addition, 5′ AMP-activated protein kinase (AMPK), which is upstream of the mTOR pathway and negatively regulates its activity, is upregulated in T_{M} cells. As shown herein, metabolic inhibition during cell activation (MIdCA), through inhibition of mTOR and/or activation of AMPK, results in CD4+ T naïve and memory cell death as well as anergy, thus leading to the depletion of pathogenic microbiota-specific CD4+ T cells. The results provided herein demonstrate that metabolic inhibition during microbiota-specific CD4⁺ T cell activation is an effective method to eliminate a pathogenic CD4⁺ T cell reservoir and induce Treg cells that provide antigen-specific and bystander suppression.

Provided herein are methods for treating or preventing inflammatory bowel disease. The methods comprise administering to a subject having inflammatory bowel disease or at risk of developing inflammatory bowel disease (a) an effective amount of a polypeptide comprising one or more flagellin T-cell receptor (TCR) epitopes; and (b) an effective amount of an agent that reduces flagellin antigen-specific memory T cells and/or increases regulatory T cells in the subject.

Also provided are methods for delaying or reducing the intensity of a relapse or flare of an inflammatory bowel disease in a subject. The methods comprise administering to a subject (a) an effective amount of a polypeptide comprising one or more flagellin T-cell receptor (TCR) epitopes; and (b) an effective amount of an agent that reduces flagellin antigen-specific memory T cells and/or increases regulatory T cells in the subject.

As used throughout, inflammatory bowel disease (IBD) is a group of intestinal disorders that cause chronic or prolonged inflammation of the digestive tract. Inflammation can occur anywhere along the digestive tract, for example, in the mouth, esophagus, stomach, small intestine and/or large intestine. Examples of inflammatory bowel disease include, but are not limited to, Crohn's disease and ulcerative colitis. In Crohn's disease, the condition most commonly affects the small intestine and colong, but it can occur anywhere in the gastrointestinal tract. Ulcerative colitis is typically limited to the colon, i.e, the large intestine.

As used herein, the terms, polypeptide, peptide, and protein are used interchangeably herein to refer to a polymer of amino acid residues. As used herein, the terms encompass amino acid chains of any length, including full-length proteins, wherein the amino acid residues are linked by covalent peptide bonds.

As used herein, a T cell receptor epitope is a peptide that can be recognized by T-cell receptors after a particular antigen has been intracellularly processed, bound to at least one MHC molecule and expressed on the surface of an antigen presenting cell as a MHC-peptide complex.

In some methods, the polypepitde comprising one or more flagellin TCR epitopes comprises one or more microbiota flagellin TCR epitopes. In some methods, the one or more flagellin TCR epitopes included in the polypeptide are TCR epitopes selected from one or more flagellins selected from the group consisting of R. inulinivorans, R. hominis, R. faecis, Eubacteria rectale, R. intestinalis, (Agathobacter rectalis) and a Lachnospiracae flagellin. In some methods the Lachospiracae flagellin is selected from the group consisting of Lachnospiraceae Flax, Lachnospiraceae 14-2, Lachnospiraceae A4 and Lachnospiraceae CBir1. In some methods, one or more flagellin TCR epitopes included in the polypeptide are from human microbiota. In some methods one or more flagellin TCR epitopes included in the polypeptide are from murine microbiota.

In some methods, the polypeptides provided herein comprise, consist of, or consist essentially of, one or more flagellin TCR epitopes comprising SEQ ID NO: 1 (DMATEMVKYSNANILSQAGQ). In some methods, the polypeptide provided herein further comprise SEQ ID NO: 2 (ISQAVHAAHAEINEAGR). In some methods, the polypeptide further comprises SEQ ID NO: 3 (EAWGALANWAVDSA). An example of a polypeptide comprising SEQ ID NO: 1, SEQ ID NO: 2 and SEQ ID NO: 3 is SEQ ID NO: 4 (MRGSHHHHHHGSMRKQIRGLTQASTNAEDGISSVQTAEGALTEVHDMLQRMNELAIQ AANGTDMATEMVKYSNANILSQAGQDMATEMVKYSNANILSQAGQDMATEMVKYSN ANILSQAGQISQAVHAAHAEINEAGREAWGALANWAVDSARGSHHHHHH). Another example of a multi-epitope polypeptide that can be used in the methods provided herein is SEQ ID NO: 5

(MVVQHNMQAMNANRMLNVTTLTEVHSMLQRMNELAVQASNGMVVQHNM
TAANANRMGETHSILQRMNELATQAANMVVQHNLTAMNANRQLVGTTGM
VVQHNMQAANANRMLGITSVHSMLQRMNELAVQAASNGTNSMVVQHNMQ
AANANRMLNVTTLTEVHSMLQRMNELATQSANGLTEVHSMLQRMNELAV
QSSNGDMAEEMVEYSKNNILAQAGQSMLAQANQSMAEEMVNYSKNNILA
AQAGQSMLAQANQMAKEMVNYSKNNILAQAGQSMLAQANDMAEEMVTYS
KNNILAQAGQSMLAQANQMVVQHNLRAMNSNRMLGITQSAQRSLLGAVQ
NRLEHTINNNEAHSILQRMNELAVQGANDVEYSKNNILAQAGQSMLAQA
NQMVVQHNLRAMNSNRMLSITQDMATEMVKFSNSNILAQAGQMVVQHNL
RAMNANRMLGITTEVHDMLQRMNELAVKAAN).

SEQ ID NO: 5 comprises SEQ ID NO: 6 (MVVQHNMQAMNANRMLNVTT), SEQ ID NO: 7 (LTEVHSMLQRMNELAVQASNG), SEQ ID NO: 8 (MVVQHNMTAANANRM), and SEQ ID NO: 9 (GETHSILQRMNELATQAAN), as set forth in Table 1.

Other, non-limiting examples of flagellin TCR epitopes that can be included in any of the multi-epitope polypeptides described herein are set forth in Table 1. Table 1 indicates the source of the individual peptides shown, for example, as shown above in SEQ ID NO: 5, which is a multiepitope peptide that can be used as a therapeutic agent. The epitopes shown in Table 1 can be linked directly to one another in the order as they are listed in Table 1 for example, SEQ ID NO: 6-SEQ ID NO: 27). These epitope peptides can also be spaced apart with linkers, or used in a different order.

In some examples, the polypeptide comprises SEQ ID NO: 10 (MVVQHNLTAMNANRQLVGTTG), derived from R. hominis, as set fort in Table 1. In some examples, the polypeptide comprises SEQ ID NO: 37 (MVVQHNMQAANANRMLGITS), derived from R. faecis, as set forth in Table 1. In some examples, the polypeptide comprises SEQ ID NO: 11 (MVVQHNMQAANANRMLNVTT), SEQ ID NO: 12 (LTEVHSMLQRMNELATQSANG) and/or SEQ ID NO: 13 (LTEVHSMLQRMNELAVQSSNG) derived from Eubacteria rectale, as set fort in Table 1. In some examples, the polypeptide comprises SEQ ID NO: 14 (DMAEEMVEYSKNNILAQAGQSMLAQANQS), derived from R. hominis, R. inulinivorans,or R. intestinalis, as shown in Table 1. In some examples, the polypepitde comprises SEQ ID NO: 15 (MAEEMVNYSKNNILAAQAGQSMLAQANQ), derived from R. inulinivorans, or Eubacteria rectale, as shown in Table 1. In some examples, the polypeptide comprises SEQ ID NO: 16 (MAKEMVNYSKNNILAQAGQSMLAQAN), derived from R. faecis or Eubacteria rectale, as shown in Table 1. In some examples, the polypeptide comprises SEQ ID NO: 17 (DMAEEMVTYSKNNILAQAGQSMLAQANQ), derived from R. intestinalis or R. inulinivorans, as shown in Table 1. In some examples, the polypeptide comprises SEQ ID NO: 18 (MVVQHNLRAMNSNRMLGITQ) and/or SEQ ID NO: 19 (SAQRSLLGAVQNRLEHTINN), derived from Lachnospiracae Flax, as shown in Table 1. In some examples, the polypeptide comprises SEQ ID NO: 20 (NEAHSILQRMNELAVQGAND) and/or SEQ ID NO: 21 (VEYSKNNILAQAGQMLAQANQ), derived from Lachnospiracae 14-2, as shown in Table 1. In some examples, the polypeptide comprises SEQ ID NO: 22 (MVVQHNLRAMNSNRMLSITQ) and/or SEQ ID NO: 23 (DMATEMVKFSNSNILAQAGQ), derived from Lachnospiracae A4, as shown in Table 1. In some examples, the polypeptide comprises SEQ ID NO: 24 (MVVQHNLRAMNANRMLGIT) and/or SEQ ID NO: 25 (TEVHDMLQRMNELAVKAAN), derived from Lachnospiracae A4, as shown in Table 1. In other examples, a polypeptide comprising SEQ ID NO: 26 (MKVKVLSLLVPALLVAGAAN) and/or SEQ ID NO: 27 (VDVGATYYFNKNMSTYVDYK), can be administered. In some examples, the polypeptide comprises one or more, two or more, three or more, four or more, five or more, six or more, seven or more, eight or more, nine or more, ten or more, eleven or more, twelve or more, thirteen or more, fourteen or more, fifteen or more, sixteen or more, seventeen or more, eighteen or more, nineteen or more, or twenty or more peptide epitopes selected from the group consisting of SEQ ID NO: 6, SEQ ID NO: 7 SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26 and SEQ ID NO; 27. In some examples, the polypeptide comprises SEQ ID NO: 6-SEQ ID NO: 27. Optionally, one or more copies of each epitope peptide can be included in the polypeptide.

In some embodiments, any of the polypeptides described herein can be conjugated to a heterologous moiety. The heterologous moiety can be, e.g., a heterologous polypeptide, a therapeutic agent (e.g., a toxin or a drug), or a detectable label such as, but not limited to, a radioactive label, an enzymatic label, a fluorescent label, a heavy metal label, a luminescent label, or an affinity tag such as biotin or streptavidin. Suitable heterologous polypeptides include, e.g., an antigenic tag (e.g., FLAG (DYKDDDDK) (SEQ ID NO:31), polyhistidine (6-His; HHHHHH) (SEQ ID NO:32), hemagglutinin (HA; YPYDVPDYA) (SEQ ID NO: 33), glutathione-S-transferase (GST), or maltose-binding protein (MBP)) for use in purifying the polypeptides.

Optionally, a heterologous peptide tag comprising or consisting of SEQ ID NO: 28 (MRGSHHHHHHGMASMTGGQQMGRDLYDDDDKDHPFT) can be linked or conjugated to any of the polypeptides described herein. As set forth above, a polyhistidine tag can be used It is understood that any polypeptide set forth herein comprising a polyhistidine tag is also provided as a polypeptide that does not comprise a polyhistidine tag (for example, SEQ ID NO: 32) at the N-terminus or the C-terminus of the polypeptide.

Heterologous polypeptides also include polypeptides (e.g., enzymes) that are useful as diagnostic or detectable markers, for example, luciferase, a fluorescent protein (e.g., green fluorescent protein (GFP)), or chloramphenicol acetyl transferase (CAT). Suitable radioactive labels include, e.g., ³²P, ³³P, ¹⁴C, ¹²⁵I, ¹³¹I, ³⁵S, and ³H. Suitable fluorescent labels include, without limitation, fluorescein, fluorescein isothiocyanate (FITC), green fluorescent protein (GFP), DyLight™ 488, phycoerythrin (PE), propidium iodide (PI), PerCP, PE-Alexa Fluor® 700, Cy5, allophycocyanin, and Cy7. Luminescent labels include, e.g., any of a variety of luminescent lanthanide (e.g., europium or terbium) chelates. For example, suitable europium chelates include the europium chelate of diethylene triamine pentaacetic acid (DTPA) or tetraazacyclododecane-1,4,7,10-tetraacetic acid (DOTA). Enzymatic labels include, e.g., alkaline phosphatase, CAT, luciferase, and horseradish peroxidase.

In some methods, the multi-epitope polypeptide comprises one or more TCR epitopes comprising the same flagellin amino acid sequence, for example, one, two, three, four, five, six or more TCR epitopes comprising the same flagellin sequence. In some methods, the multi-epitope polypeptide comprises two or more TCR epitopes wherein at least one of the TCR epitopes comprises a different flagellin sequence, for example, the polypeptide can comprise two, three, four, five, six or more TCR epitopes where at least one of the TCR epitopes comprises a different flagellin sequence as compared to the other TCR epitopes in the polypeptide. The TCR epitopes can be sequentially linked with or without linker sequence in between the TCR epitopes. Linker sequences of two, three, four, five, six or more amino acids can be used to link the TCR epitopes in any of the polypeptides described herein.

Provided herein is a polypeptide comprising one or more TCR epitopes selected from the group consisting of SEQ ID NO: 1, SEQ ID NO: 2, SEQ ID NO: 3, SEQ ID NO: 6, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 34, SEQ ID NO: 35, SEQ ID NO: 36 and SEQ ID NO: 37. Optionally, the polypeptide is not a full-length flagellin polypeptide sequence. Polypeptides having at least about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity with any of the polypeptides described herein are also provided.

Nucleic acids encoding any of the polypeptides provided herein are also provided. The term nucleic acid or polynucleotide, refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). The term nucleic acid is used interchangeably with gene, cDNA, and mRNA encoded by a gene.

Vectors, including viral and non-viral vectors comprising any of the nucleic acid sequences provided herein are also provided. Cells comprising any of the nucleic acids described herein are also provided.

TABLE 1
Flagellin peptide TCR epitopes of human microbiota

| | |
|---|---|
| Sequence | SEQ ID NOS |

AMINO DOMAIN

| | | | | |
|---|---|---|---|---|
| R. inulinivorans | Fla2 | 1-20 | MVVQHNMQAMNANRMLNVTT | 6 |
| | | 80-100 | LTEVHSMLQRMNELAVQASNG | 7 |
| | Fla5 | 1-15 | MVVQHNMTAANANRM | 8 |
| | Fla5 | 81-99 | GETHSILQRMMNELATQAAN | 9 |
| R. hominis | Fla1 | 1-20 | MVVQHNLTAMNANRQLVGTTG | 10 |
| R. faecis | Fla1, Fla3 | 1-20 | MVVQHNMQAANANRMLGITS | 37 |
| | | 83-102 | VHSMLQRMNELAVQAASNGTNS | 38 |
| Eubacteria rectale | Fla1, Fla2 | 1-20 | MVVQHNMQAANANRMLNVTT | 11 |
| (Agathobacter | Fla1, Fla3 | 80-100 | LTEVHSMLQRMNELATQSANG | 12 |
| rectalis) | Fla2 | 80-100 | LTEVHSMLQRMNELAVQSSNG | 13 |

CARBOXY DOMAIN

| | | | | |
|---|---|---|---|---|
| R. hominis | Fla1 | 243-268 | DMAEEMVEYSKNNILAQAGQSMLAQANQS | 14 |
| R. inulinivorans | Fla1 | 227-255 | | |
| R. intestinalis | Fla2 | 242-269 | | |
| R. inulinivorans | Fla2, Fla4 | 241-268 | MAEEMVNYSKNNILAAQAGQSMLAQANQ | 15 |
| Eubacteria rectale | Fla1 | 240-267 | | |
| R. faecis | Fla1, Fla3 | 243-270 | MAKEMVNYSKNNILAQAGQSMLAQAN | 16 |
| Eubacteria rectale | Fla2 | 241-268 | | |
| R. intestinalis | Fla1 | 198-230 | DMAEEMVTYSKNNILAQAGQSMLAQANQ | 17 |
| R. inulinivorans | Fla5 | | | |

Flagellin peptide TCR epitopes of murine microbiota

| | | | | |
|---|---|---|---|---|
| Lachnospiraceae FlaX | FlaX | 1-20 | MVVQHNLRAMNSNRMLGITQ | 18 |
| | | 384-403 | SAQRSLLGAVQNRLEHTINN | 19 |
| Lachnospiraceae 14-2 | Fla1 | 81-100 | NEAHSILQRMNELAVQGAND | 20 |
| | | 354-375 | VEYSKNNILAQAGQSMLAQANQ | 21 |
| Lachnospiraceae A4 | Fla4 | 1-20 | MVVQHNLRAMNSNRMLSITQ | 22 |
| | | 417-436 | DMATEMVKFSNSNILAQAGQ | 23 |
| Lachnospiraceae A4 | Fla3 | 1-19 | MVVQHNLRAMNANRMLGIT | 24 |
| | | 81-99 | TEVHDMLQRMNELAVKAAN | 25 |

The term identity, as used in the context of polynucleotide or polypeptide sequences, refers to a sequence that has at least 80% sequence identity to a reference sequence. Alternatively, percent identity can be any integer from 80% to 100%. Exemplary embodiments include at least: 80%, 85%, 90%, 95%, or 99% identity, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like. For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A comparison window, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Math. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215:403-410 and Altschul et al. (1977) Nucleic Acids Res. 25:3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P (N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

In the methods provided herein, the polypeptide comprising one or more TCR epitopes, i.e., a multi-epitope polypeptide, activates flagellin-specific T cells. Activation of T cells refers to any treatment or manipulation of T cells which results in an increase (i.e., enhancement, upregulation, induction, stimulation) in the number, biological activity and/or survivability of the T cells. Therefore, increasing the activity of T cells can be accomplished by increasing the number of T cells in a subject (i.e., by causing the cells to proliferate/expand or by recruiting additional T cells to a site), increasing a type of T cell in a subject relative to another type of T cell, for example, increasing the number of regulatory T cells relative to one or more other types of T cells in the subject, by increasing the activation of T cells in an animal, by increasing biological activity of T cells (e.g., effector functions or other activities of the cell) in an animal and/or by increasing the ability of T cells to survive in a subject. In the methods provided herein, an increase in T cell activation can be an increase of at least about 10%, 20%, 30%, 50%, 60%, 70%, 80%, 90%, 100%, 200%, 300%, 400% or greater as compared to a control, for example, T cell activation in the absence of the polypeptide comprising one or more TCR epitopes.

As used herein, the phrase, T cell, refers to a lymphoid cell that expresses a T cell receptor molecule. T cells include human alpha beta (αβ) T cells and human gamma delta (γδ) T cells. T cells include, but are not limited to, naïve T cells, stimulated T cells, primary T cells (e.g., uncultured), helper T cells, cytotoxic T cells, memory T cells, regulatory T cells, natural killer T cells, combinations thereof, or sub-populations thereof. T cells can be CD4⁺, CD8⁺, or CD4⁺ and CD8⁺. T cells can also be CD4⁻, CD8⁻, or CD4⁻ and CD8⁻ T cells can be helper cells, for example helper cells of type T_{H}1, T_{H}2, T_{H}3, T_{H}9, T_{H}17, or T_{FH}. T cells can be cytotoxic T cells. Regulatory T cells can be FOXP3⁺ or FOXP3⁻. In some cases, the T cell is a CD4⁺CD25^{hi}CD127^{lo} regulatory T cell. In some cases, the T cell is a regulatory T cell selected from the group consisting of type 1 regulatory (Tr1), T_{H}3, CD8+CD28−, Treg17, and Qa-1 restricted T cells, or a combination or sub-population thereof. In some methods, the polypeptide comprising one or more TCR epitopes activates flagellin-specific CD4⁺ T cells. In some methods, the polypeptide comprising one or more TCR epitopes activates flagellin-specific CD4⁺ memory T cells.

In some embodiments, the agent that reduces flagellin antigen-specific memory T cells and/or increases regulatory T cells in the subject, increases regulatory T cells by activing regulatory T cells. In some embodiments, the agent that activates or increases regulatory T cells is a mutant IL-2 polypeptide, for example, SEQ ID NO: 29 (APTSSSTKKTQLQLEHLLLDLQMILNGINNYKNPKLTRMLTFKFYMPKKATELKHLQCL EEELKPLEEALNLAPSKNFHIRPRDLISDINVIVLELKGSETTFMCEYADETATIVEFLNR WITFSQSIISTLT) or SEQ ID NO: 30 (APTSSSTKKTQLQLEHLLLDLQMILNGINNYKNPKLTRMLTFKFYMPKKATELKHLQCL EEELKPLEEALNLAPSKNFHLRPRDLISDINVIVLELKGSETTFMCEYADETATIVEFINR WITFSQSIISTLT). Other exampled include mutant IL-2 polypeptide comprising one or more substitutions selected from the group consisting of aV69A, Q74P, L80I, N88D, L118I, and a C125S substitution. Non-limiting examples of mutant IL-2 polypeptides can be found in U.S. Pat. Nos. 10,174,092, 10,174,092, 9,580,486, 7,105,653, 9,616,105, and 9,428,567, all of which are incorporated in their entireties by this reference. In some embodiments, the IL-2 mutant polypeptide further comprises an Fc peptide. In some embodiments, the Fc peptide is at the C-terminus. In some embodiments, the Fc peptide is at the N-terminus. Examples of Fc peptides can be found in U.S. Pat. Nos. 10,174,091 and 10,174,092 (See, for example, SEQ ID NO: 8, SEQ ID NO: 9 or SEQ ID NO: 15 of U.S. Pat. No. 10,174,092). In some embodiments, the IL-2 mutant is administered with an another agent that increases regulatory T cells, for example, a metabolic inhibitor. In some embodiments, the IL-2 mutant is administered with an mTOR inhibitor, for example, rapamycin.

In some embodiments, the agent that reduces flagellin antigen-specific memory T cells or increases regulatory T cells in the subject is a metabolic inhibitor. In the methods provided herein the metabolic inhibitor can inactivate T cells that have been activated by contacting the T cells with the polypeptide comprising one or more TCR epitopes, for example, CD4+ T_{M} cells. Inactivation of the microbiota-flagellin T_{M} cells or inducing Treg cells via T cell receptor (TCR) stimulation and inhibition of mTORC results in a decrease of microbiota-reactive T_{M} cells, an increase in Treg cells and/or an altered ratio of Treg/T_{E} cells. In any of the methods provided herein, a decrease or reduction in memory T (T_{M}) cells, can be a reduction or decrease of at least 10%, as compared to a reference control level, or a decrease of least about 20%, or at least about 30%, or at least about 40%, or at least about 50%, or at least about 60%, or at least about 70%, or at least about 80%, or at least about 90%, or at least about 95%, or at least about 100%. A decrease or reduction in T_{M} cells can also be a decrease or reduction in the biological activity of memory T (T_{M}) cells. In any of the methods provided herein, an increase in Treg cells can be an increase of at least about 10%, as compared to a reference control level, or an increase of least about 20%, or at least about 30%, or at least about 40%, or at least about 50%, or at least about 60%, or at least about 70%, or at least about 80%, or at least about 90%, or at least about 95%, or at least about 100%, or at least about 200%, or at least about 300%, or at least about 400%. In some methods, TCR stimulation, i.e., flagellin-specific T cell activation, and mTORC inhibition occur simultaneously. In other methods, mTORC inhibition occurs subsequent to flagellin-specific T cell activation.

In some methods, the metabolic inhibitor is an FK506-binding protein 12-rapamycin-associated protein 1 (mTOR) inhibitor. Examples of mTOR inhibitors include, but are not limited to rapamycin, sirolimus, temsirolimus, everolimus, ridaforolimus, dactolisib, BGT226, SF1126, PKI-587 and sapanisertib. In other methods, an ATPase inhibitor, for example, Bz423, a pro-apoptotic 1, 4 benzodiazepine, can be administered. In the methods provided herein, the polypeptide comprising one or more TCR epitopes can be administered to the subject simultaneously with the metabolic inhibitor or prior to or after administration of the metabolic inhibitor. In some methods, the polypeptide comprising one or more TCR epitopes and the metabolic inhibitor are administered simultaneously, or shortly after administration of the polypeptide that activates flagellin specific T cells so that the metabolic inhibitor can inactivate the recently activated T cells.

Any of the methods provided herein can further comprise administering a protein kinase AMP-activated catalytic subunit alpha 1 (AMPK) activator to the subject. Examples of AMPK activators include, but are not limited to, metformin, troglitazone, prioglitazone, rosiglitazone, resveratrol, quercetin, genistein, epigallocatechin gallate, berberine, curcumin, ginsenoside Rb1, α-lipoic acid and cryptotanshinone. The AMPK activator can be administered to the subject simultaneously with, prior to or after administration of the metabolic inhibitor. The AMPK activator can be administered to the subject simultaneously with, prior to or after administration of a polypeptide comprising one or more flagellin T-cell receptor (TCR) epitopes.

Any of the methods provided herein can be performed in conjunction with other therapies for inflammatory bowel disease (combination therapy). For example, a polypeptide comprising one or more flagellin T-cell receptor (TCR) epitopes, a metabolic inhibitor and/or an AMPK activator can be administered to a subject at the same time, prior to, or after, surgery, chemotherapy, immunotherapy, gene therapy, cell transplant therapy, genome editing therapy, or other pharmacotherapy.

As used herein, the term subject means a mammalian subject. The term subject can be used interchangeably with the term patient. Exemplary subjects include, but are not limited to humans, monkeys, dogs, cats, mice, rats, cows, horses, camels, goats and sheep. In some embodiments, the subject is a human. In some embodiments, the subject has or is suspected of having an inflammatory bowel disorder, for example, Crohn's disease or ulcerative colitis. Optionally, the subject is diagnosed with an inflammatory bowel disease or at risk for developing an inflammatory bowel disease, for example, Crohn's disease or ulcerative colitis. The subject can be a human with an inflammatory bowel disease, wherein the subject has an increased anti-flagellin response, as compared to a control. The subject can be a human with an inflammatory bowel disease, wherein the subject has an increased anti-Lachnospiraceae flagellin response, as compared to a control. The subject can be a human with an inflammatory bowel disease, wherein the subject has an increased anti-Cbir 1 flagellin response, as compared to a control. Exemplary controls include, but are not limited to, a subject that is in remission, a healthy subject or a control value. In some methods, the subject can be a human subject that can be suspected of having an inflammatory bowel disease that can be treated with a polypeptide comprising one or more flagellin TCR epitopes and a metabolic inhibitor.

Optionally, a subject can be tested for immune reactivity to one or more antigens, for example, microbiota peptide sequences (for example, microbiota flagellin antigens) to identify one or more antigens for which the subject has an increased response, as compared to a control. Once the one or more antigens are identified, a polypeptide comprising the one or more antigens can be administered to the subject. Any of the methods provided herein, can further comprise administering a polypeptide comprising one or more antigens for which the subject has an increased response to the subject. In some examples, the identified one or more antigens can be included in any of the polypeptides described herein, for example, in a polypeptide comprising one or more antigens selected from the group consisting of SEQ ID NO: 1, SEQ ID NO: 2, SEQ ID NO: 3, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26 and SEQ ID NO; 27. In some examples, the one or more antigens for which the subject has an increased response can be included in a polypeptide comprising SEQ ID NO: 4 or SEQ ID NO: 5.

Treating or treatment of any disease or disorder refers to ameliorating a disease or disorder that exists in a subject. The term ameliorating refers to any therapeutically beneficial result in the treatment of a disease state, e.g., an inflamamtory bowel disease, lessening the severity or progression, promoting remission or durations of remission, or curing thereof. Thus, treating or treatment includes ameliorating at least one physical parameter or symptom. Treating or treatment includes modulating the disease or disorder, either physically (e.g., stabilization of a discernible symptom) or physiologically (e.g., stabilization of a physical parameter) or both. Treating or treatment includes delaying or preventing progression of an inflammatory bowel disease. Thus, in the disclosed methods, treatment can refer to a 10%, 20%, 30%, 40%, 50%, 60%, 70%, 80%, 90%, or 100% reduction in the severity of an established disease or condition or symptom of the disease or condition. For example, a method for treating an inflammatory bowel disease is considered to be a treatment if there is a 10% reduction in one or more symptoms of the autoimmune disorder (for example, digestive issues, abdominal pain, fatigue, skin problems, swollen glands, fever, etc.) in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition.

As used herein, a relapse or flare is considered an exacerbation of the inflammatory bowel disease that causes new symptoms or worsening of previous symptoms. Subjects who achieve remission, or symptom free periods to initial treatment and then experience a recurrence are said to have had a relapse or flare of an inflammatory bowel disease. One or more relapses may occur days, months or years after the initial remission.

### Administration

As used herein, administer or administration refers to the act of introducing, injecting or otherwise physically delivering a substance as it exists outside the body (e.g., a multi-epitope polypeptide and/or a metabolic inhibitor) into a subject, such as by mucosal, intradermal, intravenous, intramuscular, intrarectal, oral, subcutaneous delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof.

The compositions are administered in a number of ways depending on whether local or systemic treatment is desired, and on the area to be treated. The compositions are administered via any of several routes of administration, including orally, parenterally, intramucosally, intravenously, intraperitoneally, intraventricularly, intramuscularly, intradermally, subcutaneously, intracavity or transdermally. Administration can be achieved by, e.g., topical administration, local infusion, injection, or by means of an implant. The implant can be of a porous, non-porous, or gelatinous material, including membranes, such as sialastic membranes, or fibers. The implant can be configured for sustained or periodic release of the composition to the subject. See, e.g., U.S. Patent Application Publication No. 20080241223; U.S. Pat. Nos. 5,501,856; 4,863,457; and 3,710,795; and European Patent Nos. EP488401 and EP 430539. The composition can be delivered to the subject by way of an implantable device based on, e.g., diffusive, erodible, or convective systems, osmotic pumps, biodegradable implants, electrodiffusion systems, electroosmosis systems, vapor pressure pumps, electrolytic pumps, effervescent pumps, piezoelectric pumps, erosion-based systems, or electromechanical systems. In some embodiments, the multi-epitope polypeptide and the metabolic inhibitor are therapeutically delivered to a subject by way of local administration. Effective doses for any of the administration methods described herein can be extrapolated from dose-response curves derived from in vitro or animal model test systems.

As used herein, the term therapeutically effective amount or effective amount refers to an amount of a polypeptide comprising one or more flagellin TCR epitopes, a metabolic inhibitor or AMPK activator that, when administered to a subject, is effective to treat a disease or disorder either by one dose or over the course of multiple doses. A suitable dose can depend on a variety of factors including the particular polypeptide used and whether it is used concomitantly with other therapeutic agents. Other factors affecting the dose administered to the subject include, e.g., the type or severity of the inflammatory bowel disease. For example, a subject having ulcerative colitis may require administration of a different dosage of a multi-epitope polypeptide a metabolic inhibitor and/or an AMPK activator than a subject with Crohn's disease. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject also depends upon the judgment of the treating medical practitioner. A therapeutically effective amount is also one in which any toxic or detrimental effects of the composition are outweighed by the therapeutically beneficial effects.

A pharmaceutical composition can include a therapeutically effective amount of any multi-epitope polypeptide, metabolic inhibitor and/or AMPK activator described herein. In some embodiments, the pharmaceutical composition can further comprise a carrier. Such effective amounts can be readily determined by one of ordinary skill in the art as described above. Considerations include the effect of the administered multi-epitope polypeptide, or the combinatorial effect of the multi-epitope polypeptide with one or more additional active agents, if more than one agent is used in or with the pharmaceutical composition. Cell culture assays and animal studies can be used in formulating a range of dosage for use in humans.

The term carrier means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject. Such pharmaceutically acceptable carriers include sterile biocompatible pharmaceutical carriers, including, but not limited to, saline, buffered saline, artificial cerebral spinal fluid, dextrose, and water.

Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the agent described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected agent without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material known in the art for use in pharmaceutical formulations. The choice of a carrier for use in a composition will depend upon the intended route of administration for the composition. The preparation of pharmaceutically acceptable carriers and formulations containing these materials is described in, e.g., Remington: The Science and Practice of Pharmacy, 22nd edition, Loyd V. Allen et al, editors, Pharmaceutical Press (2012).

Examples of physiologically acceptable carriers include buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN® (ICI, Inc.; Bridgewater, New Jersey), polyethylene glycol (PEG), and PLURONICS™ (BASF; Florham Park, NJ).

Compositions containing the agent(s) described herein suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof are admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

The agents described herein can be incorporated into pharmaceutical compositions which allow for immediate release or delivery of those agents to a mammal. The agents described herein can also be incorporated into pharmaceutical compositions which allow for modified release, for example, delayed release or extended release (for example, sustained release or controlled release) of those agents to a mammal for a period of several days, several weeks, or a month or more. Such formulations are described, for example, in U.S. Pat. Nos. 5,968,895 and 6,180,608 and are otherwise known in the art. Any pharmaceutically-acceptable, delayed release or sustained-release formulation known in the art is contemplated.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, such as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

In some examples, a nucleic acid is employed, for example, a nucleic acid encoding encoding a polypeptide comprising one or more flagellin T-cell receptor (TCR) epitopes and/or a nucleic acid encoding the mutant IL-2 polpeptide is administered to the subject. The nucleic acid can be delivered with a carrier. Nucleic acid carriers include, polyethylene glycol (PEG), PEG-liposomes, branched carriers composed of histidine and lysine (HK polymers), chitosan-thiamine pyrophosphate carriers, surfactants, nanochitosan carriers, and D5W solution. The present disclosure includes all forms of nucleic acid delivery, including synthetic nucleic acids, naked DNA, plasmid and viral delivery, integrated into the genome or not.

As mentioned above, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The exact method is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naidini et al., Science 272:263-267, 1996), and pseudotyped retroviral vectors (Agrawal et al., Exper. Hematol. 24:738-747, 1996). The nucleic acid can also be encapsulated in a nanoparticle or chemically conjugated to a carrier. For example, the nucleic acid can be chemically conjugated to a cell or a tissue-targeting ligand, such as an antibody or a ligand for a cell-surface receptor to target nucleic acid to specific cell types or tissue environments. Physical transduction techniques can also be used, such as liposome delivery and receptor-mediated and other endocytosis mechanisms (see, for example, Schwartzenberger et al., Blood 87:472-478, 1996) to name a few examples. Nucleic acid delivery can local or systemic, via any of the delivery methods described herein, for example, and not to be limiting, via oral, parenteral, intramucosal, intravenous, intraperitoneal, intraventricular, intramuscular, subcutaneous, intracavity or transdermal administration.

Disclosed are materials, compositions, and ingredients that can be used for, can be used in conjunction with or can be used in preparation for the disclosed embodiments. These and other materials are disclosed herein, and it is understood that when combinations, subsets, interactions, groups, etc. of these materials are disclosed that while specific reference of each various individual and collective combinations and permutations of these compositions may not be explicitly disclosed, each is specifically contemplated and described herein. For example, if a method is disclosed and discussed, and a number of modifications that can be made to a number of molecules included in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Microbiota Antigen CD4 T Memory Cells (T_{M})

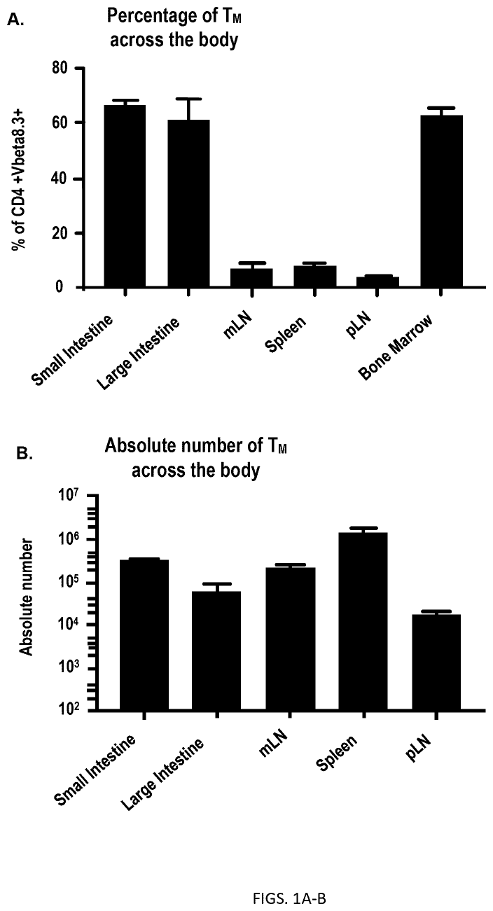

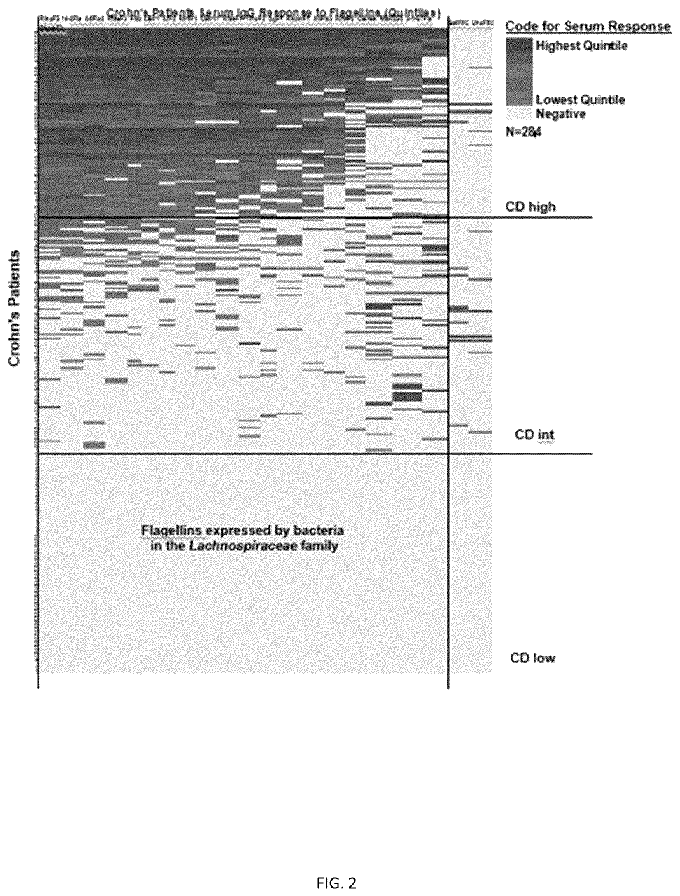

CD4+ T cell effectors mediate colitis in mouse models. These effectors, for example, Th1 and Th17 are short-lived. The CD4+ T cell memory pool is a reservoir of chronicity and regenerating T effector cells. CD4 T_{M} cells are widely distributed in CBir1 TCR Tg mice (FIG. 1). Flagellin specific T cells can cause murine colitis, mainly Th17 mediated. Anti-CBir1 was detected in 50% of Crohn's patients and in multiple mouse models. The presence of anti-Cbir1 in CD patients is associated with small bowel (SB) involvement, fibrostenosis and internal perforating phenotypes, independent of anti-Saccharomyces cerevisiae antibodies (ASCA), anti-12 antibodies and anti-OmpC antibodies. A subset of Crohn's patients has broad IgG reactivity to microbiota flagellins (FIG. 2). Flagellins have promiscuous T cell receptor epitopes. Therefore, whether microbiota flagellins contain epitope peptides that are broadly reactive in humans and can stimulate both T_{M} and Tregs across a population of patients with Crohn's disease was studied. Peptides predicted to bind to 8 or more HLA-DRB1 molecules and to mouse H-2^{b} or H-2^{d} were studied. Thirty predicted flagellin epitopes were tested for binding to the four most common HLA-DR dimers (HLA-DR3, HLA-DR7, HLA-DR11 and HLA-DR15). All predicted flagellin epitopes bound at least one of the four HLA-DR dimers, and twenty of the predicted epitopes bound two or more. Table 2 shows the binding of flagellin peptides to HLA-DR.

TABLE 2

| | | EC50 | Ratio* | |
|---|---|---|---|---|
| | DR0301 | DR0701 | DR1101 | DR1501 |
| CBir1 p456-475 | — | 3.7 | — | 0.32 |
| R. Intest. Fla1 p408-427 | 55 | 5.0 | 58 | 4.1 |
| FlaX p1-20 | — | — | — | 0.33 |
| A4 Fla3 p1-19 | — | 2.5 | 0.2 | 0.32 |
| E. rectale Fla1 p445-464 | — | — | 0.3 | 4.5 |

*<20 = high affinity 20-100 + moderate affinity
All 30 peptides bound at least one of the four HLA-DRs
19/20 peptides bound 2 or more of the four HLA-DRs

Table 3 shows predicted TCR epitopes for CBir-1. Polypeptides comprising any of these peptides can be used in the methods provided here. Cbir-1 polypeptide (#1-17) is SEQ ID NO: 34. Cbir-1 polypeptide (#411-426) is SEQ ID NO: 35. Cbir-1 polypeptide (#456-475) is SEQ ID NO: 36.

TABLE 3

| CBir-1 | | | | | | | | | | | | |
|---|---|---|---|---|---|---|---|---|---|---|---|---|
| peptide | | HLA-DRB1* | | | | Percentile rank† | | | | H-2 | | |
| # | Sequence | 01:01 | 03:01 | 04:01 | 04:05 | 07:01 | 08:02 | 09:01 | 11:01 | 13:01 | 15:01 | b/d |
| 1-17 | MVVQHNLQAMN | 4.8 | 4.1 | 0.2 | 2.7 | 5.1 | 8.6 | 3.8 | 4.6 | 3.1 | 2.3 | + |
| | SNRMLG | | | | | | | | | | | |
| 411-426 | AGAIKKVSTQR | 9.8 | 6.0 | 2.6 | 7.2 | 1.9 | 0.1 | — | 4.7 | 19.5 | 16.4 | + |
| | SALGA | | | | | | | | | | | |
| 456-475 | MATEMVKYSNA | 17.0 | 4.6 | 2.5 | 7.0 | 0.8 | 0.7 | 1.6 | 3.9 | 0.2 | 0.8 | + |
| | NILSQAGQ | | | | | | | | | | | |

†Percentile rank: the lower the rank the better the binding of the peptide to HLA-DR; Scores above 20 percentile considered as non-binding;
+, peptide predicted to bind with high affinity to either H-2^{b} or H-2^{d}

T cells undergo reprogramming as they differentiate. Proliferating T cells have to regenerate the entire cell (proteins, lipids, nucleotides) in about 1 day. Proliferating T cells use aerobic glycolysis for anabolic regeneration. mTOR kinase regulates anabolic cell metabolism. mTOR and AMPK are key regulator for cell metabolism.

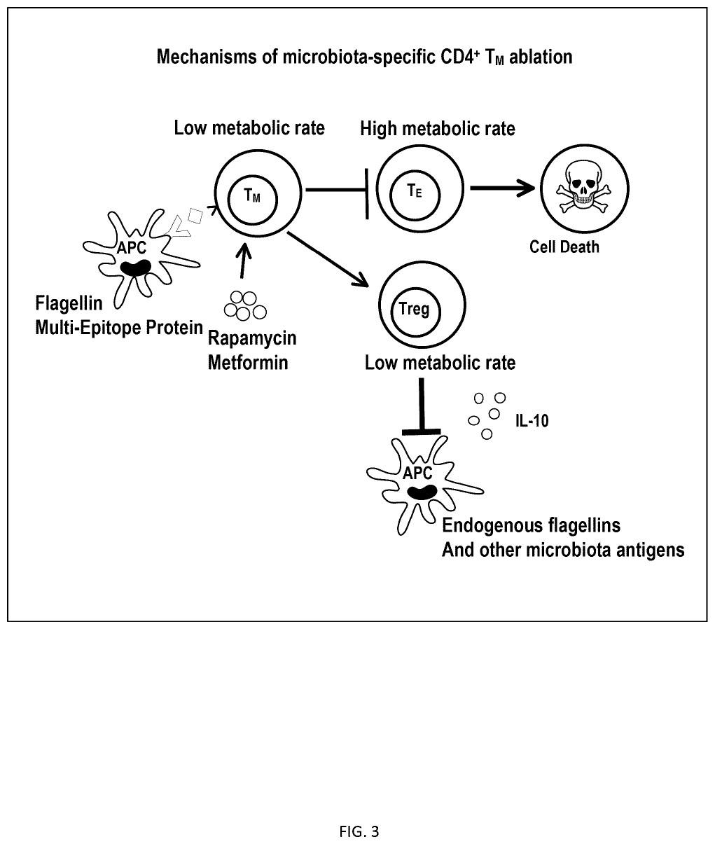

It was hypothesized that inactivating the microbiota-flagellin T_{M} cells or transforming them into Treg cells via simultaneous T cell receptor (TCR) stimulation and inhibition of mTORC would result in the ablation of microbiota-reactive TM cells and an altered ratio of Treg/TE. A model for this process is shown in FIG. 3.

### Rapamycin Inhibits Microbiota-Specific CD4+ naïve T Cell Activation and Proliferation Through Metabolic Targeting In Vitro

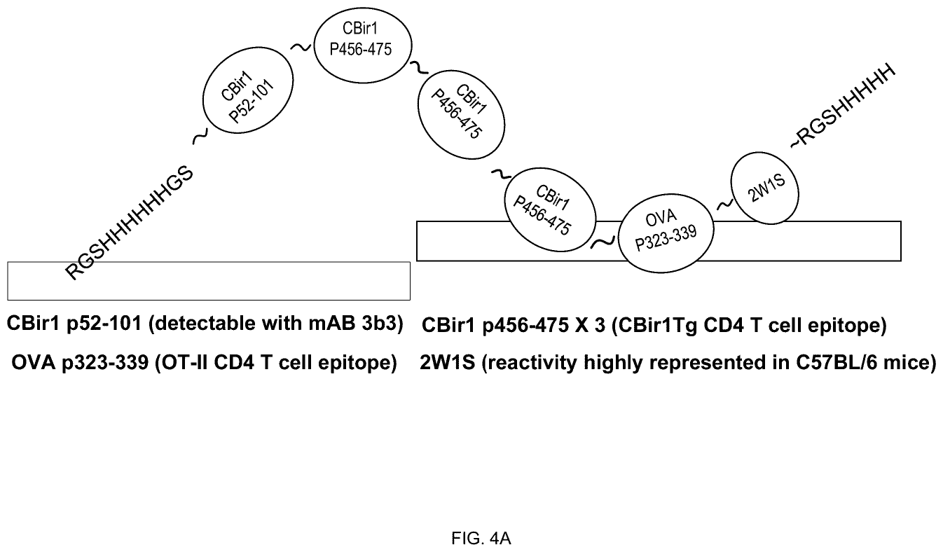

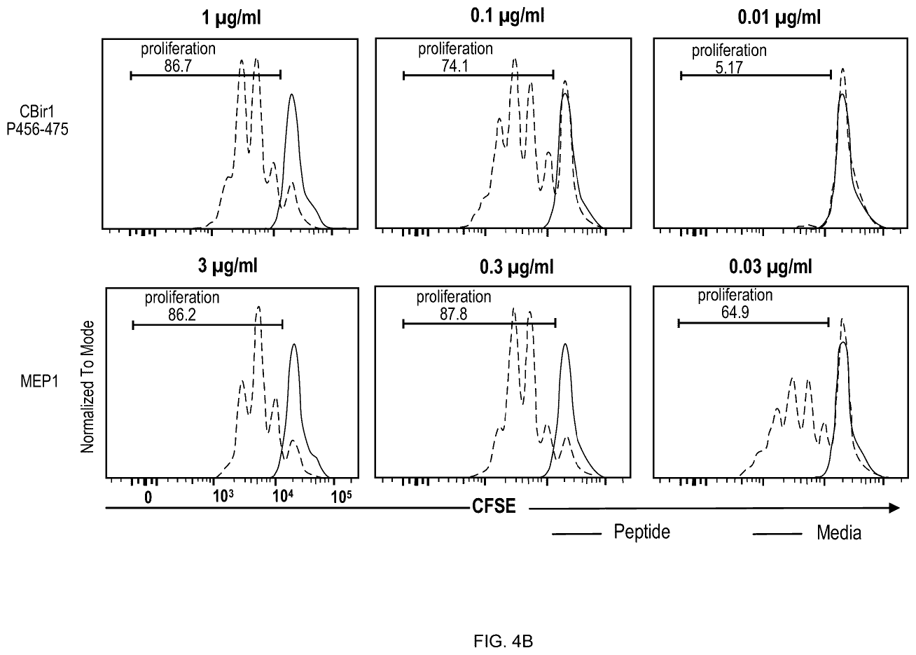

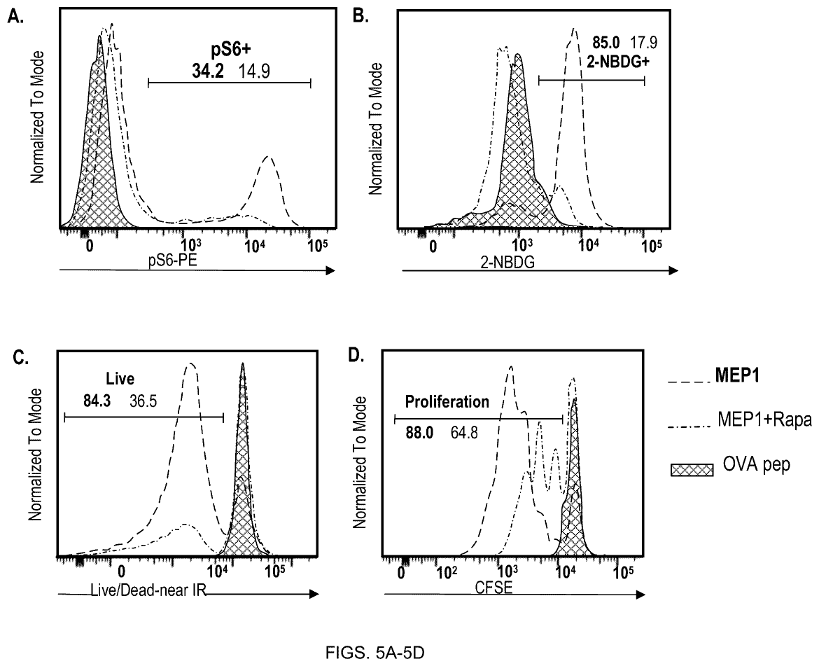

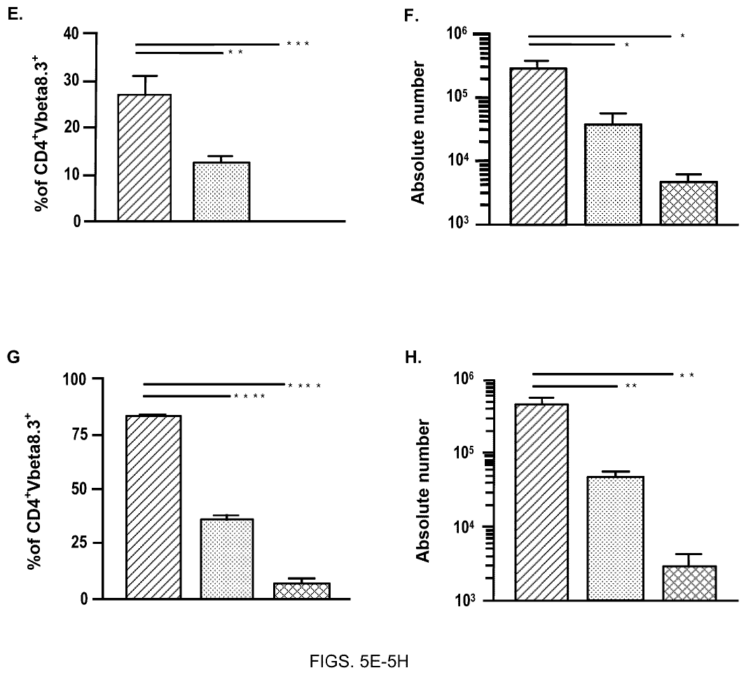

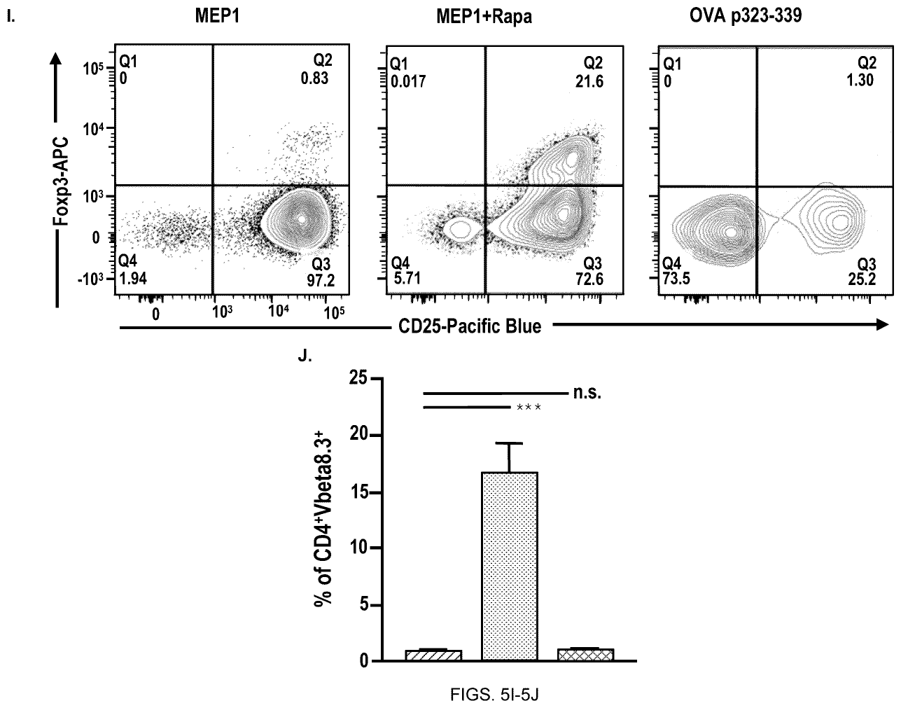

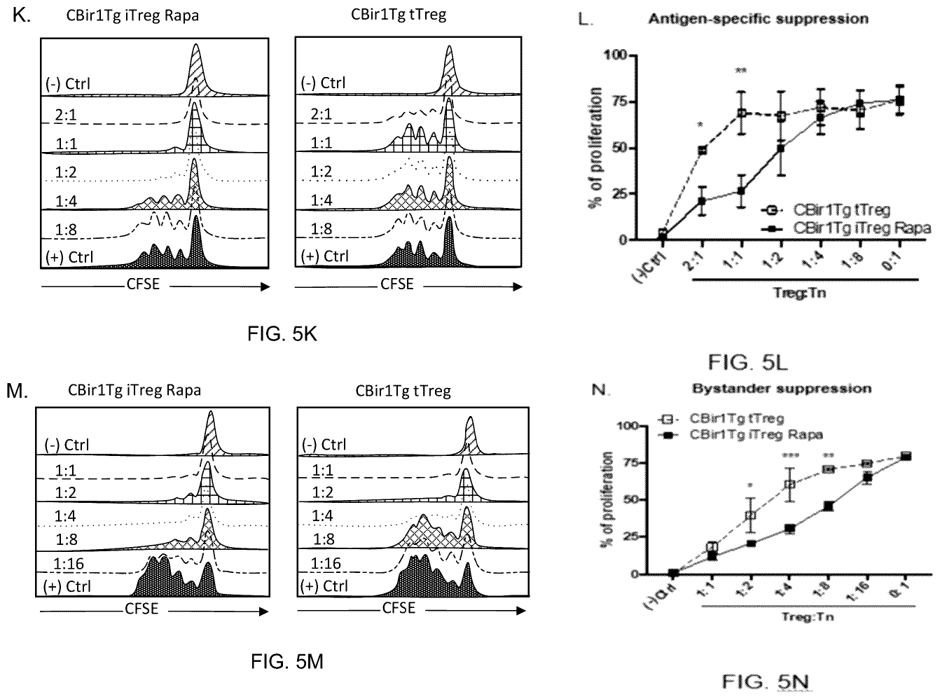

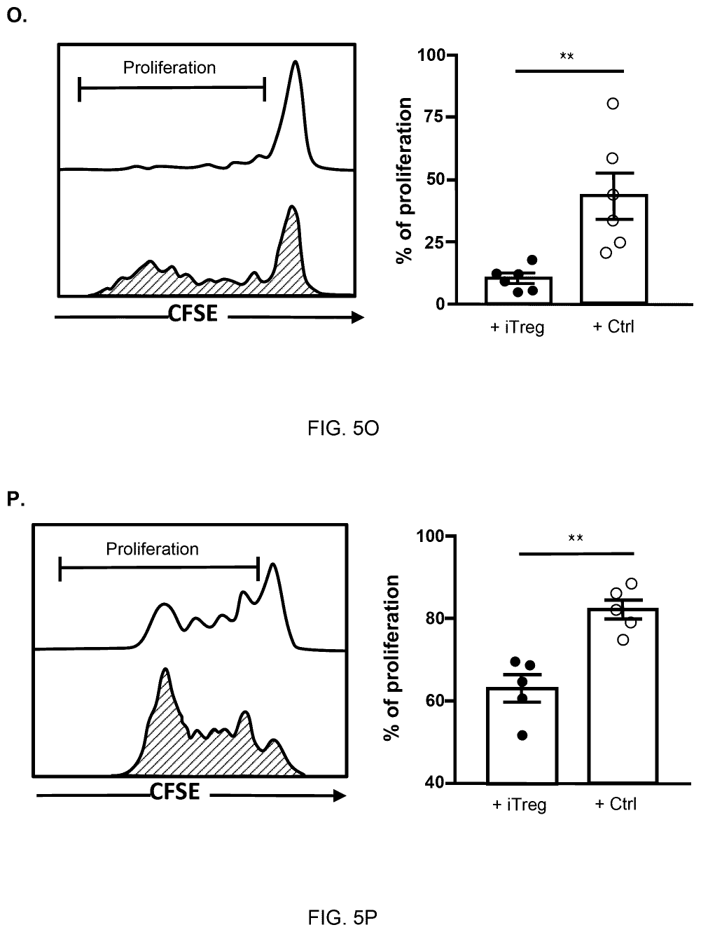

To perform the studies described herein, a multi-epitope-peptide-1 (MEP1) construct, which included three repeats of CBir1 TCR Tg CD4+ T cell epitope (CBir1 p456-479), and one repeat of OT-II Tg CD4+ T cell epitope (OVA p323-339 (ISQAVHAAHAEINEAGR) (SEQ ID NO: 2), was engineered. Dose response comparisons with corresponding peptides confirmed that antigen presenting cells (APCs) were able to process and present MEP1 at a comparable, or even higher level, as compared to single peptides (FIGS. 4A and 4B). Then, whether rapamycin has any impact on CD4+ naïve T cell metabolism and function, when it is simultaneously applied upon cognate antigen encounter, was tested. In the presence of 100 nM rapamycin, the phosphorylation of mTORC's main downstream target S6 kinase (S6K) resulted in roughly 60% reduction when CBir1Tg.CD4+CD44− naïve T cells were co-cultured with irradiated APCs and MEP1 after 20 hrs (FIGS. 5A and 5E). Correspondingly, after 90 hrs of co-culture, rapamycin treated CD4+ T cells had significantly compromised capacity for glucose metabolism, as assessed by the uptake of fluorescent glucose analog 2-NBDG (FIGS. 5B and 5F). Rapamycin also led to dramatic CBir1Tg naïve CD4+ T cell death and roughly 10-fold reduction of cell proliferation in the presence of cognate antigen MEP1 (FIGS. 5C-D and 5G-H). Of note, rapamycin has to be present at the same time as antigen encounter to achieve all of the inhibitory effects, thus demonstrating that metabolic inhibition during cell activation is a viable strategy. These results indicate that simultaneous application of rapamycin during CD4+ T cell initial cognate antigen encounter blocks the major metabolic fuel for cell activation and proliferation, in specific glucose metabolism, thus resulting in drastic antigen-specific T cell killing instead of expansion.

### Rapamycin Promotes Microbiota-Specific Treg Development, With Enhanced Suppressive Function In Vitro and In Vivo

Unlike activated CD4+ T cells, Treg cells mainly engage fatty acid oxidation as their energy source, and maintain a low level of metabolism, which is comparable to naïve CD4⁺ cells. Thus, rapamycin has minimum impact on Treg metabolism, and in fact, rapamycin promotes substantial Treg differentiation. Compared to cells stimulated with MEP1 alone, CBir1Tg naïve CD4⁺ T cells co-cultured with MEP1 and rapamycin for 96 hrs had a 20-fold increase of Treg cells in percentage (FIGS. 5I and 5J). To test if rapamycin-induced Treg cells are functionally suppressive in vitro and in vivo, naïve CD4⁺ T cells isolated from CBir1Tg.Foxp3gfp mice were used, so that GFP⁺CD4⁺CD25⁺ Treg cells could be sorted out after induction with MEP1 and rapamycin for 5 days (CBir1Tg iTreg Rapa). GFP+CD4+CD25+ cells freshly isolated from these mice were used as control Tregs (CBir1Tg tTreg) for an in vitro suppression assay. Naïve CD4⁺ T cells isolated from congenic CBir1Tg or C57BL/6 mice were labelled with proliferation dye and stimulated with CBir1 p456-479 or anti-CD3, respectively, in the presence of the indicated ratios of rapamycin-induced Tregs or fresh ex vivo Tregs, to test their response under antigen-specific (FIGS. 5K and 5L) or bystander (FIGS. 5M and 5N) suppression. In both manners, it was found that rapamycin-induced Tregs strongly suppressed the proliferation of responder cells, and they showed a better suppression than fresh isolated Treg cells. Rapamycin-induced Treg cells were also able to provide antigen-specific and bystander suppressions in vivo.

Equal amounts of CBir1Tg iTreg Rapa and CFSE labelled congenic naïve CD4+ responder cells (CBir1Tg or OT-II) were co-transferred into C57BL/6 mice, and the recipient mice were challenged with CBir1 p456-479 or MEP1, respectively. Mice transferred with CBir1Tg naïve CD4+ T cells plus responder cells, that received the same challenge, served as the corresponding control group. The proliferation of responder cells was inhibited in both manners when CBir1Tg iTreg Rapa cells were present (FIGS. 5O and 5P). These data showed that rapamycin favors the induction of phenotypic and functional Treg differentiation in addition to metabolic inhibition of other helper T cell subsets, thus providing the potential for both antigen-specific and bystander suppression of other responding CD4+ T cells.

### Simultaneous Rapamycin Treatment With Peripheral Antigen Activation Prevents the Development of Naïve CD4+ T Cell Mediated Colitis in Immunocompromised Mice

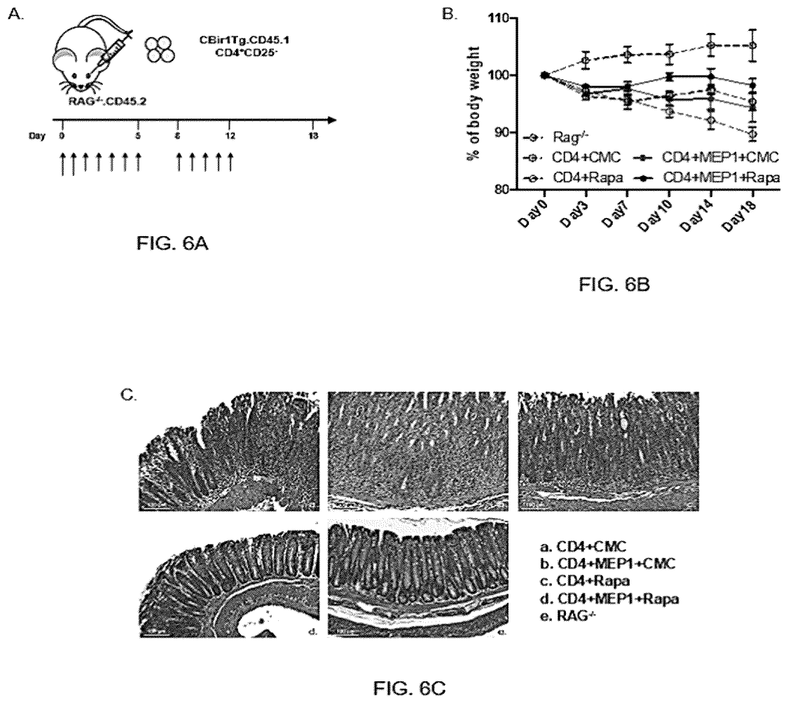

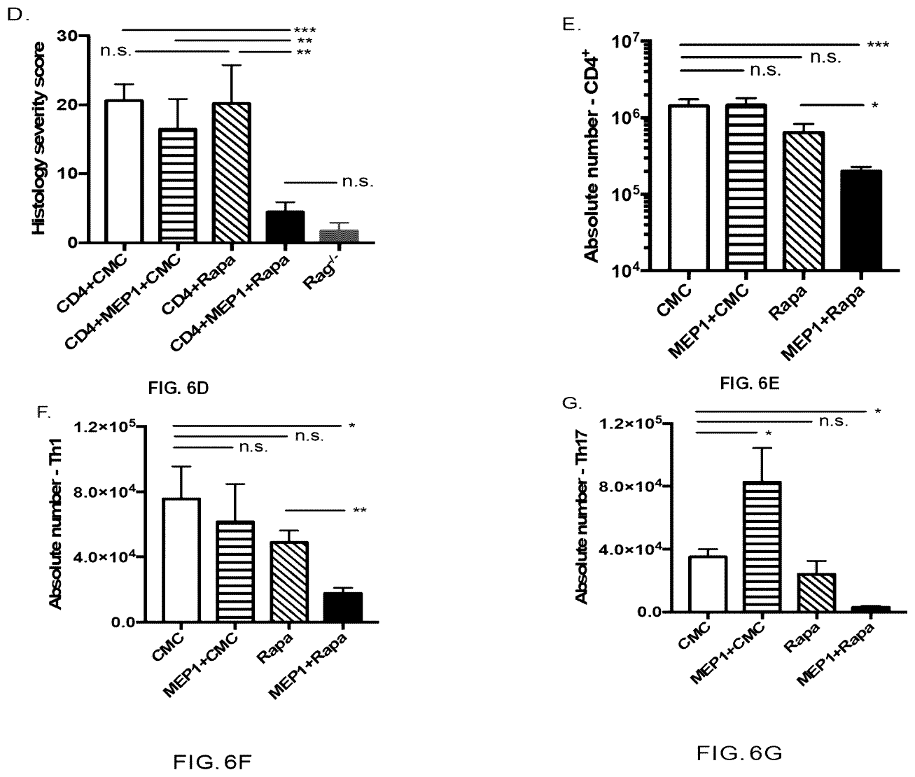

Based on the in vitro and in vivo data above, it was hypothesized that metabolic inhibition with rapamycin during microbiota antigen-specific CD4⁺ T cell activation would suppress the proliferation of effector cells, thus limiting the intensity of intestinal inflammation. However, the environment in the intestine is heavily suppressive due to constant presence of IL-10 and TGF-β, under which circumstances the inhibitory effect of rapamycin might be impeded. Therefore, it was postulated that microbiota antigen-specific cells need to be attracted to the periphery. This mimics the dissemination of microbial antigens during the disruption of the intestinal mucosa, thus allowing for better metabolic inhibition by rapamycin. To test this hypothesis, an adoptive transfer model of naïve CBir1Tg CD4⁺ T cells into Rag-/- mice was used to induce T cell mediated colitis with or without peripheral activation by MEP1. On days 1-5 and 8-12, the recipient mice were treated with rapamycin or vehicle control (0.2% CMC) intraperitoneally (i.p.) daily, and then mice were sacrificed on day 18 for histological and cellular assessment (FIG. 6A). Although weight loss was not the best reflection for the severity of colitis, especially when disease course was fairly short in the experimental setup, mice receiving peripheral MEP1 activation and rapamycin treatment had the least wasting symptoms compared to all of the other recipient groups (FIG. 6B). Correspondingly, histological assessment showed that Rag-/- mice receiving CD4⁺ cells, with and without peripheral MEP1 activation, both developed severe colitis; rapamycin treatment, but without peripheral MEP1, was not able to prevent colitis development. However, with the combination of peripheral MEP1 activation and rapamycin treatment, the development of CD4⁺ T cell mediated colitis was fully prevented, with a comparable histological appearance and disease severity score of the control, healthy Rag-/- mice (FIGS. 6C and 6D). This was because of the successful metabolic inhibition of CBir1Tg CD4⁺ cells in the periphery, resulting in significantly fewer CBir1Tg CD4⁺ cells (FIG. 6E), and, in particular, fewer pathologic effector Th1 and Th17 cells in the colon of the recipient mice as compared to other groups (FIGS. 6F and 6G). Therefore, simultaneous application of rapamycin with microbiota antigen-specific CD4⁺ T cell peripheral activation could serve as a preventative immunotherapy for the restraint of intestinal inflammation when naïve CD4⁺ T cells encounter their cognate microbiota antigens for the first time.

### Rapamycin Prevents the Development of CD4⁺ T_{M} Cell Response, but Favors the Differentiation of Treg Cells In Vivo, in an Antigen-Specific Manner

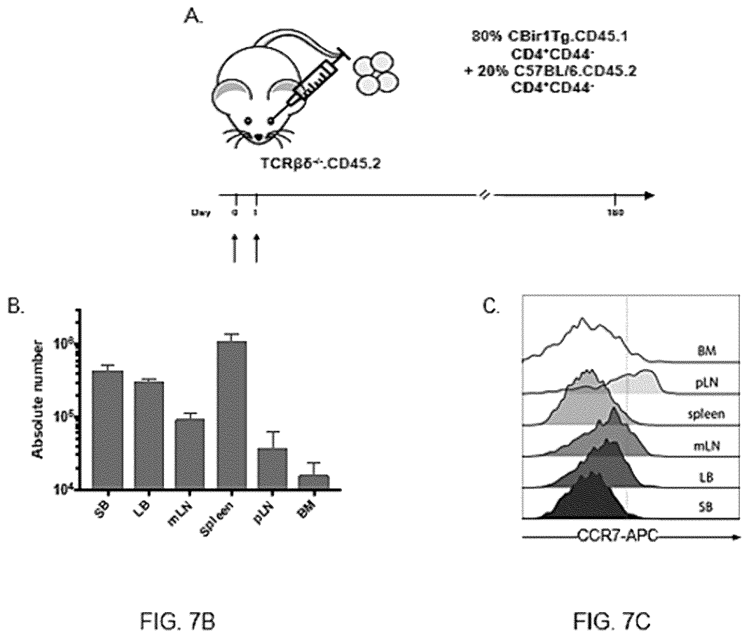

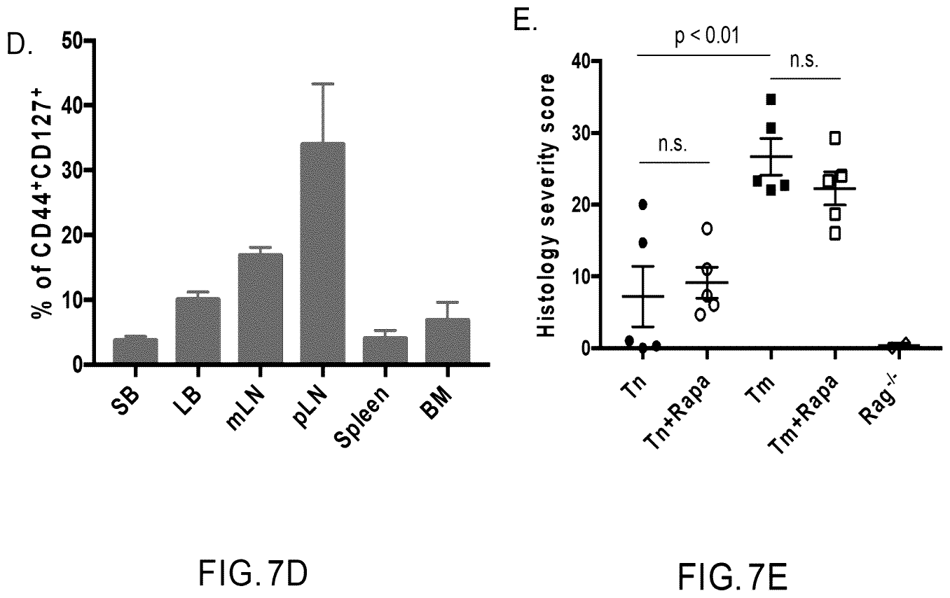

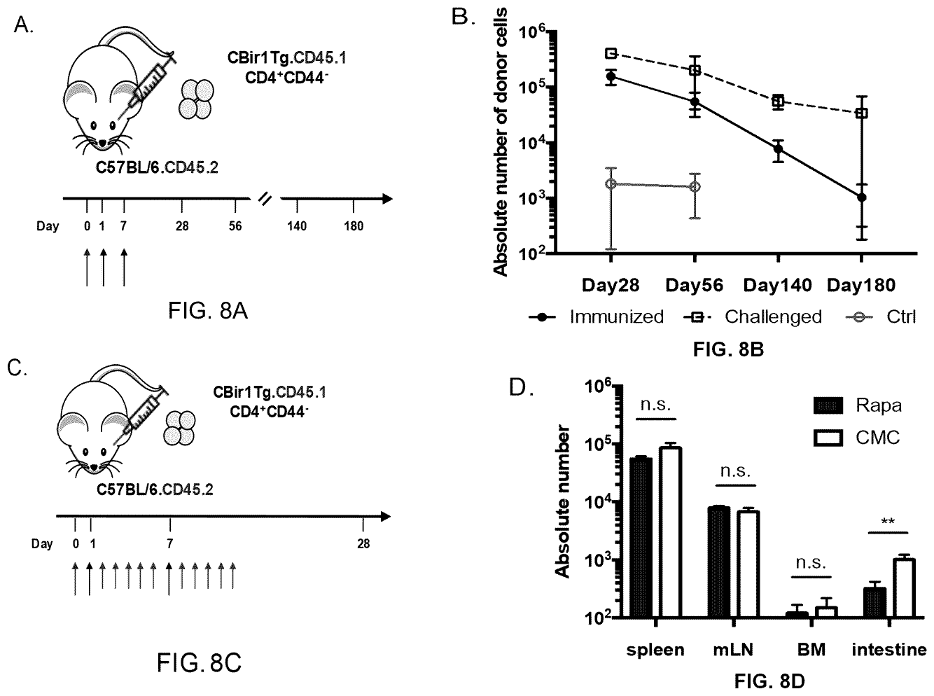

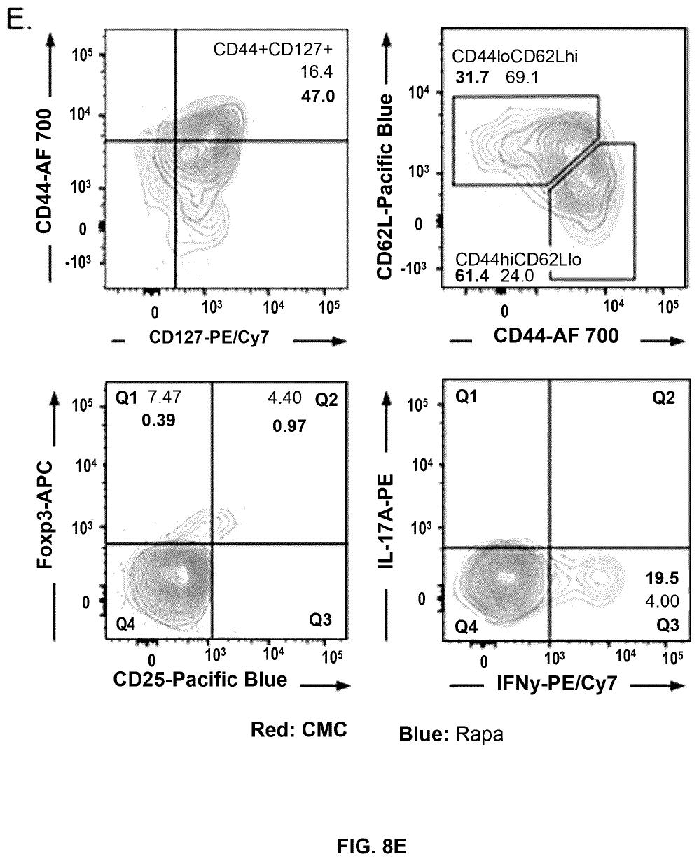

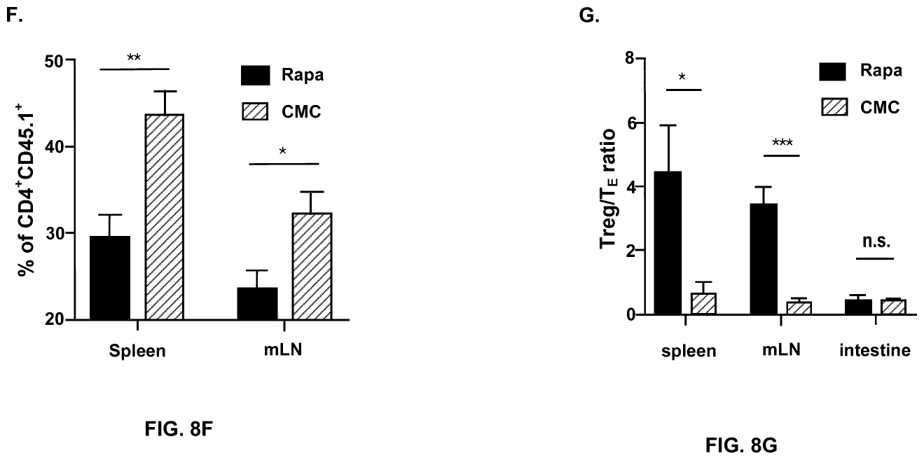

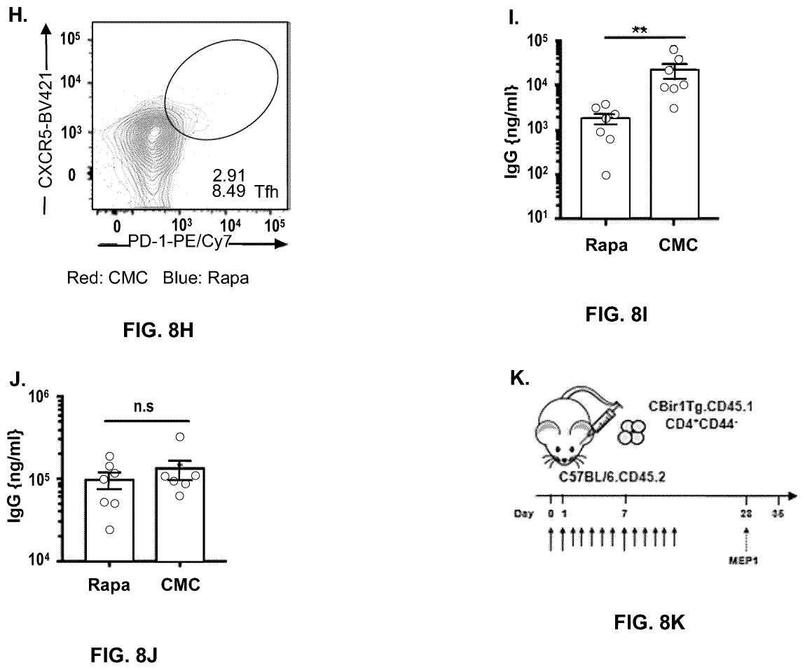

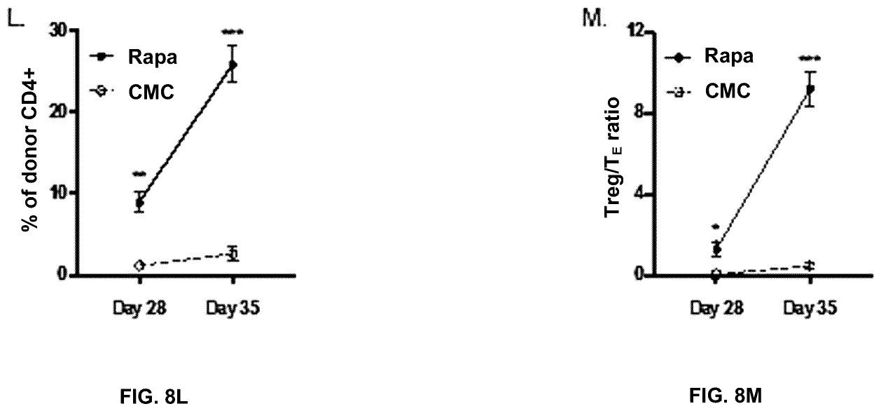

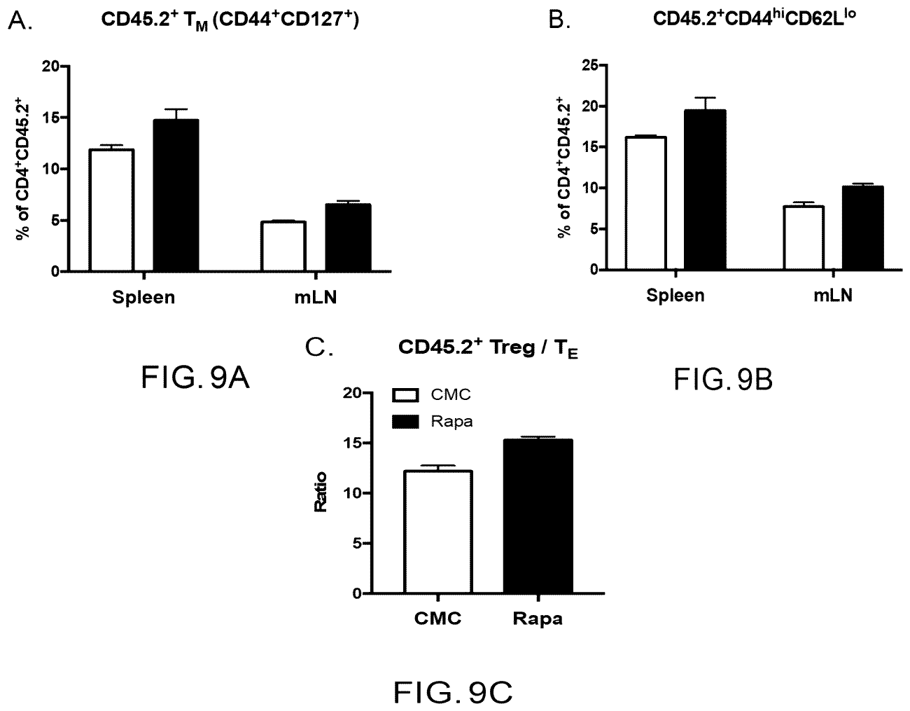

During acute gastroenteric infection, low levels of CD4⁺ T cell response against the gut microbiota could also be elicited and thus form long-lasting memory responses. Data provided herein showed that microbiota flagellin-specific T_{M} cells can also be induced during colitis, using the CBir1Tg naïve CD4⁺ T cells transfer model, and that these cells circulate through the intestine, bone marrow, and secondary lymphoid organs (FIGS. 7A-7E). Therefore, preventing the formation of microbiota-reactive CD4+ T_{M} cells with rapamycin during initial antigen encounter diminishes the possibility of upcoming intestinal inflammation. To test this hypothesis, the physiologic development of microbiota-reactive CD4⁺ TM response in the periphery was mimicked by immunizing C57BL/6 mice, which were transferred with CBir1Tg.CD45.1 CD4⁺CD44⁻ cells, twice with 50 μg CBir1 flagellin i.p. (FIG. 8A). The majority of the transferred CD4⁺ T cells expressed memory markers (CD44⁺CD127⁺, and CD62Llow) 3 weeks post immunization, and they were present in the recipient mice up to 6 months post immunization for rapid expansion upon CBir1 flagellin challenge (FIG. 8B). Then, whether simultaneous rapamycin treatment during immunization had any impact on the development of memory CD4⁺ response was analyzed. C57BL/6 mice transferred with congenic CBir1Tg naïve CD4⁺ T cells received 5 days of rapamycin injection i.p. following immunization, whereas mice receiving drug vehicle 0.2% CMC were used as controls (FIG. 8C). At day 28, the absolute numbers of transferred CD4⁺ cells in both groups were not significantly different, except that fewer CBir1Tg CD4⁺ T cells were recovered from the intestines of rapamycin-treated mice (FIG. 8D). Compared to the control group, CBir1Tg CD4⁺ T cells isolated from the spleen and mLN of rapamycin treated mice displayed less memory phenotype but more of a naïve status, by lower expression of CD44 and CD127 (FIG. 8E and 8F), and higher expression of CD62L (FIG. 8E). In the meanwhile, rapamycin treatment facilitated the expression of Foxp3, but inhibited effector cytokines in transferred cells (FIG. 8E), resulting in dramatic differences of the Treg/Teff ratio in the spleen and mLN of the recipient mice compared to the control group (FIG. 8G). Rapamycin treatment also inhibited the differentiation of antigen-specific T follicular helper (Tfh) cells, assessed by the expression of CXCR5 and PD-1 (FIG. 8H). Correspondingly, rapamycin treated mice had substantially lower anti-CBir1 flagellin IgG in the serum compared to control mice (FIG. 8I). All of these effects derived from rapamycin treatment were antigen-specific, because no such differences were found in the CD4+ cells of the hosts (FIGS. 9A-9C), as well as serum IgG response against cholera toxin B subunit (CTB) (FIG. 8J). In summary, rapamycin treatment during initial antigen encounter in the periphery prevents the development of antigen-specific CD4+ memory response and Tfh cell differentiation, while promoting the differentiation of Treg cells. Furthermore, when these mice were challenged with CBir1 flagellin on day 28, there was a dramatic expansion of Treg cells in the donor portion (FIG. 8L), but not Teff cells (FIG. 8M), indicating that rapamycin imprints the antigen-specific CD4⁺ T cells during initial antigen encounter, and favors their fate towards Treg cells upon antigen re-challenge.

### Combination of Rapamycin and Metformin Ablates Existing Microbiota Antigen-Specific CD4+ T_{M} Cells in the Periphery In Vitro and In Vivo

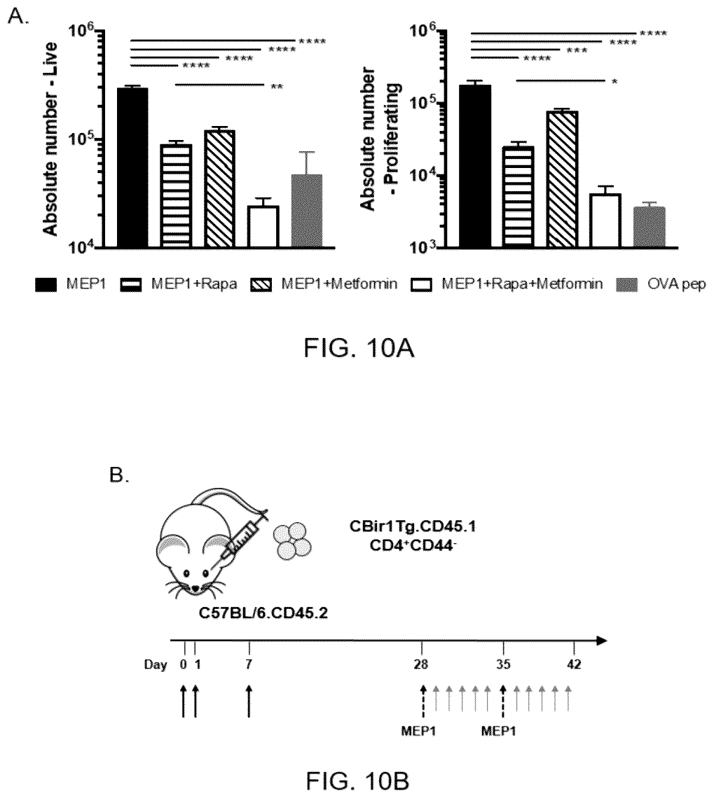

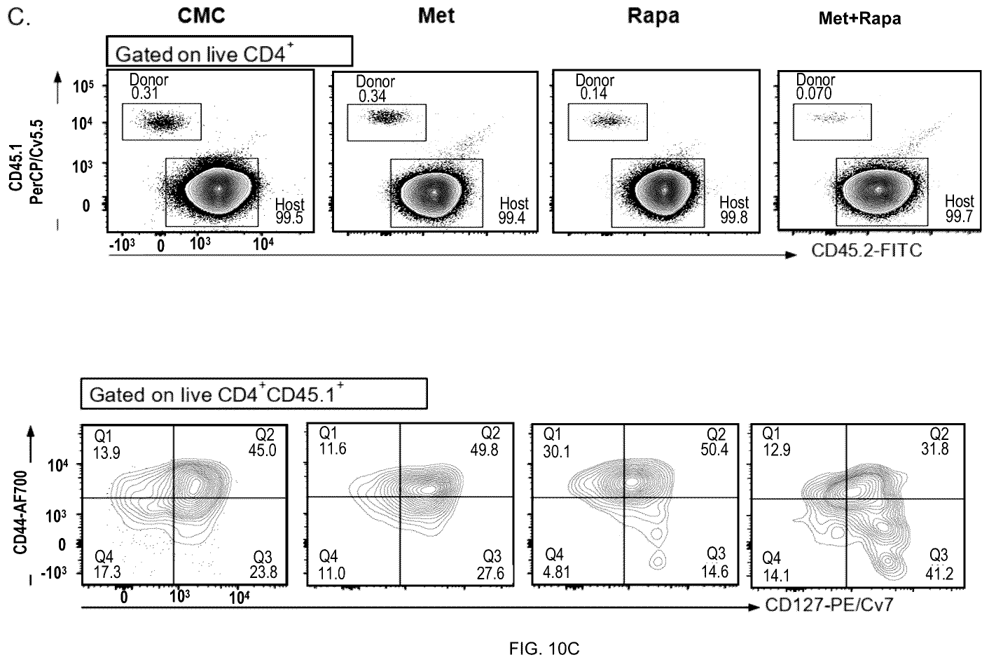

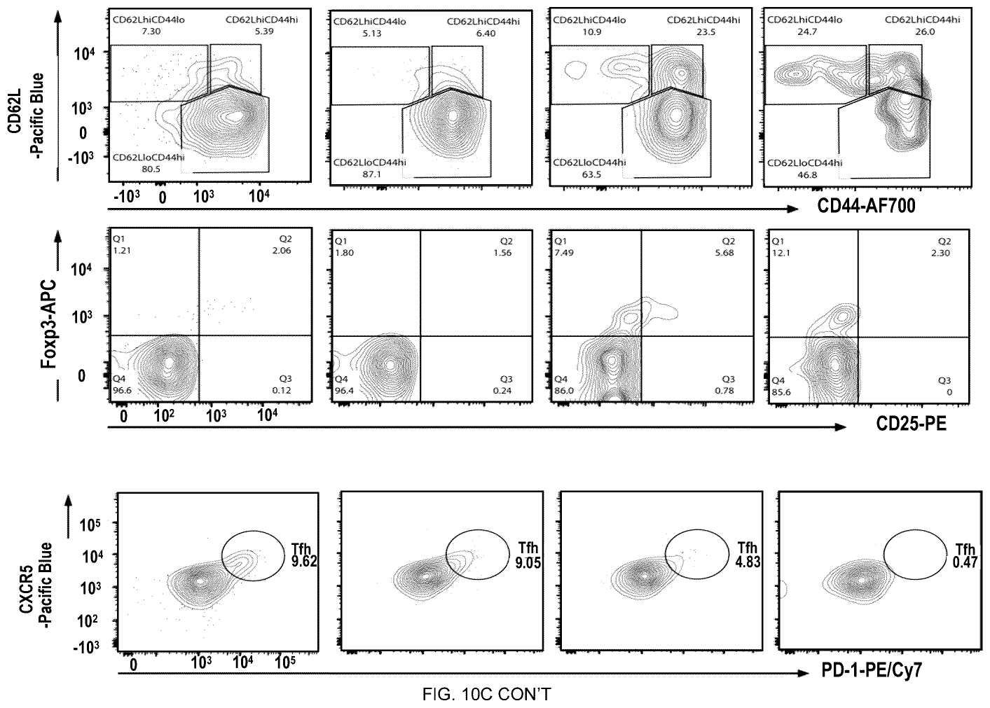

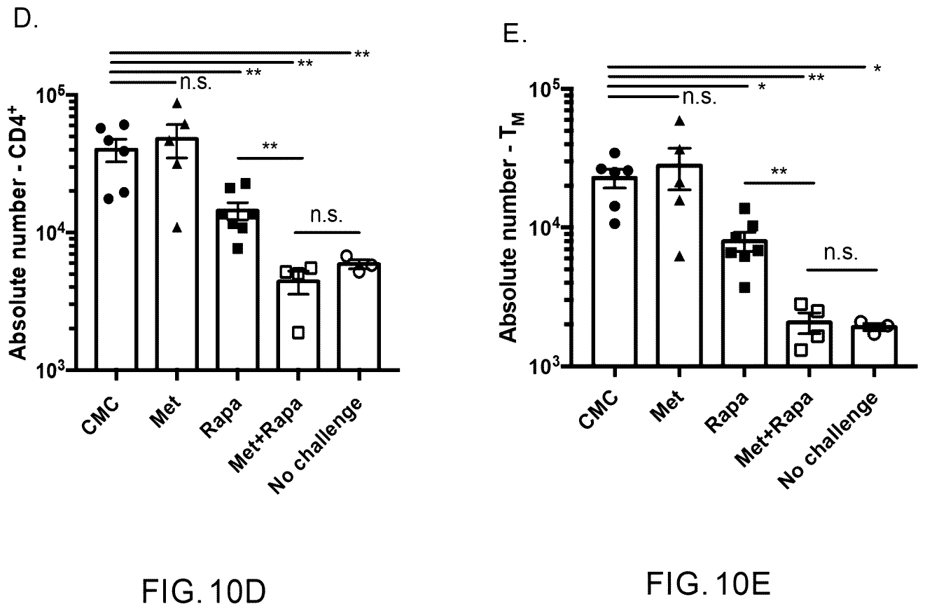

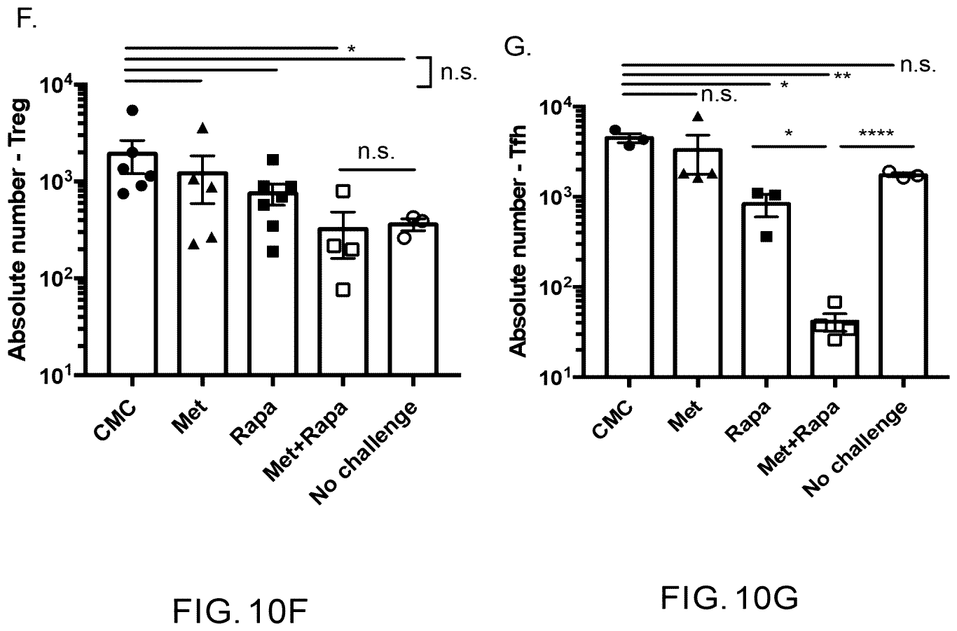

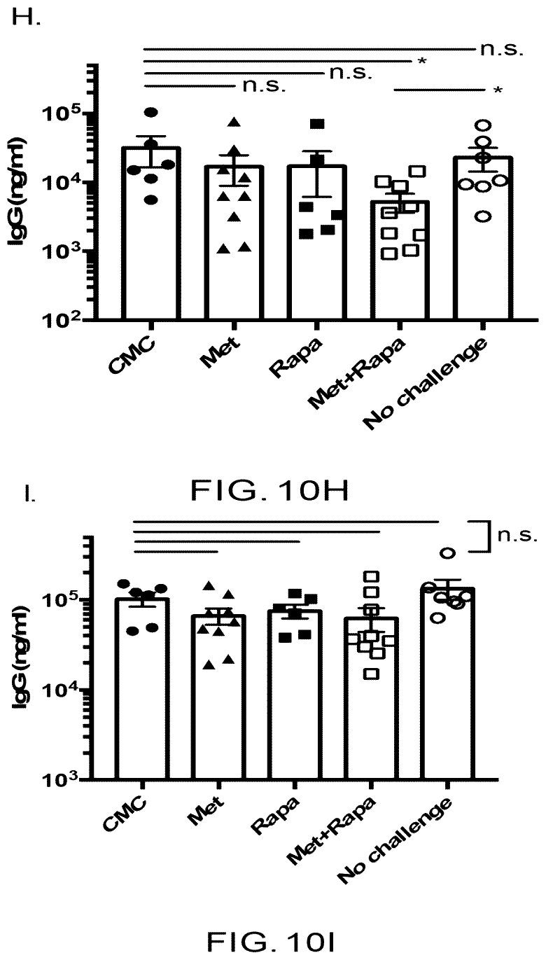

The goal for preventing the relapse of Crohn's disease is to eliminate the circulating microbiota antigen-reactive CD4⁺ T_{M} cells during disease remission. These cells are present at low frequencies in the periphery, in patients with Crohn's disease, as well as in experimental models of colitic mice. Unlike naïve CD4⁺ T cells, when CBir1Tg CD4⁺ T_{M} cells were co-cultured with MEP1 and rapamycin in vitro, significant but much less intense cell death and inhibition of cell proliferation was observed. T_{M} cells engage in minimum mTOR activity but mainly rely on upregulated AMPK activity for their metabolism. Therefore, whether the metabolic inhibitor metformin, which targets the AMPK pathway, or the combination of rapamycin and metformin, could achieve a better inhibitory effect on CD4⁺ T_{M} cells was studied. Data from in vitro culturing showed that inhibition with metformin had a comparable effect, or even worse, than rapamycin on activated CD4+ T_{M} cells. However, the combination of rapamycin and metformin showed a more robustly effective inhibition on cell survival and proliferation, compared to either when used alone (FIG. 10A). This was tested in vivo by targeting established microbiota antigen-specific CD4+ T_{M} cells in the periphery. C57BL/6.CD45.2 mice were transferred with congenic CBir1Tg CD4+ T_{N} cells and immunized with CBir1 flagellin i.p. twice for antigen-specific CD4+ T_{M} induction. Upon antigen challenge, on days 28 and 35, recipient mice were treated with metformin, rapamycin, the combination of both, or drug vehicle for 5 consecutive days, respectively. Recipient mice with immunization but no antigen challenge were used as controls (FIG. 10B). Assessed by the absolute numbers recovered from the spleen, CBir1Tg CD4+ cells vastly expanded after antigen challenge, with no inhibitory result seen when mice were treated with metformin alone. Moderate inhibition was observed with rapamycin alone. However, when mice were treated with the combination of rapamycin and metformin, the expansion of CBir1Tg CD4+ T_{M} cells was completely prohibited, which was not different from mice without challenge (FIG. 10C and 10D). Furthermore, their remaining CBir1Tg CD4+ T cells displayed a less activated phenotype, with significantly decreased co-expression of CD44 and CD127, and increased expression of CD62L (FIG. 10C and 10E). On the other hand, although the expansion of CBir1Tg CD4+ T_{M} cells was inhibited with rapamycin or rapamycin+metformin treatment, there was a significant induction of Treg population in the remaining CBir1Tg CD4⁺ cells, which was not seen in other groups (FIG. 10C and 10F). Of note, rapamycin, especially the combination of rapamycin and metformin, also inhibited the differentiation of antigen-specific Tfh cells upon challenge (FIG. 10C and 10G), resulting in a decreased level of serum anti-CBir1 flagellin IgG (FIG. 10H), whereas the level of pre-existing antibody irrelevant to antigen challenge had no difference (FIG. 10I). In summary, these results indicated that CD4⁺ T_{M} cells engage different metabolic pathways with CD4⁺ T_{N} cells. Therefore, the combination of mTOR inhibitor rapamycin and AMPK activator metformin resulted in a more robust ablation of pre-existing microbiota antigen-specific CD4⁺ T_{M} and Tfh cells, as well as induction of Treg cells.

### MIdCA With Rapamycin and Metformin Successfully Inhibits the Survival and Proliferation of Circulating Microbiota Antigen-Specific CD4+ T Cells in Crohn's Patients

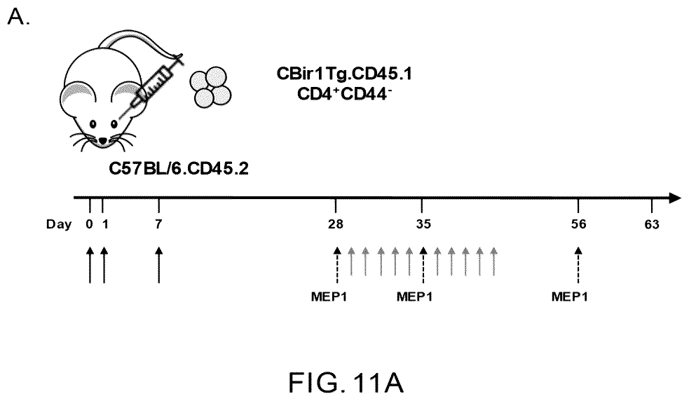

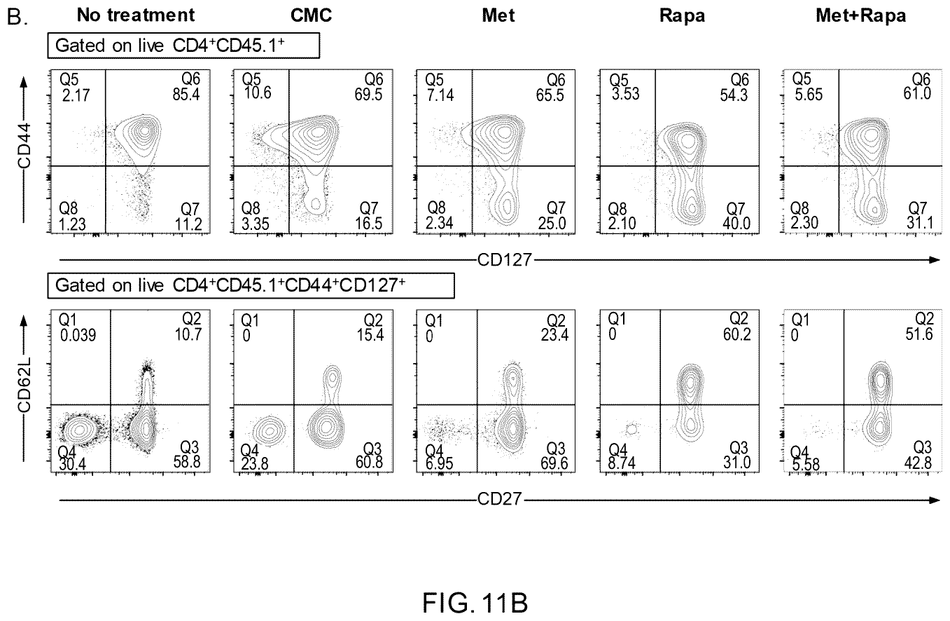

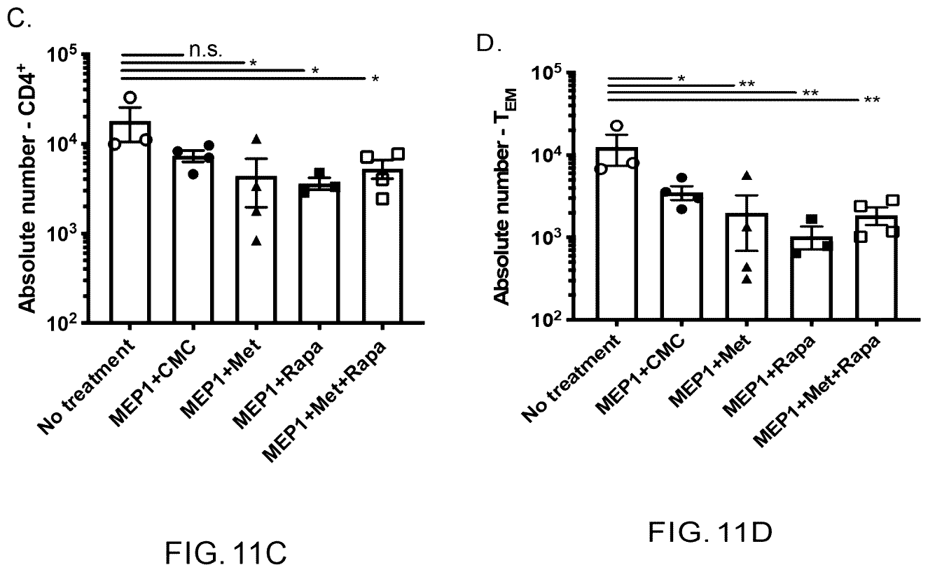

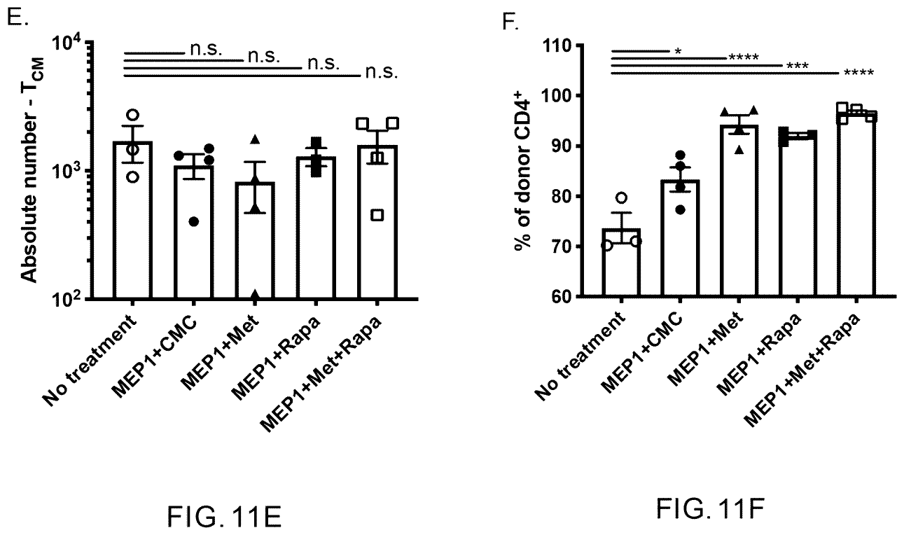

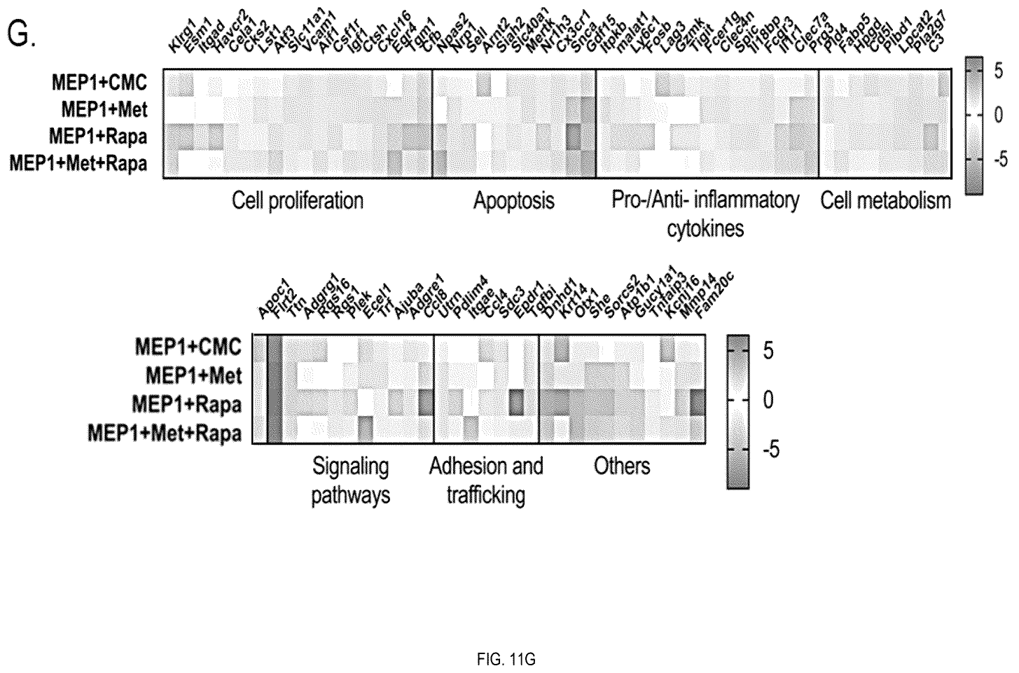

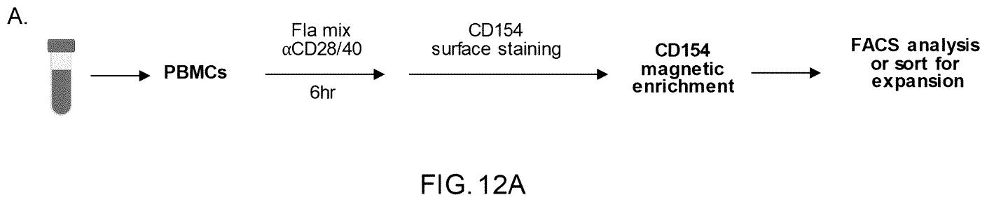

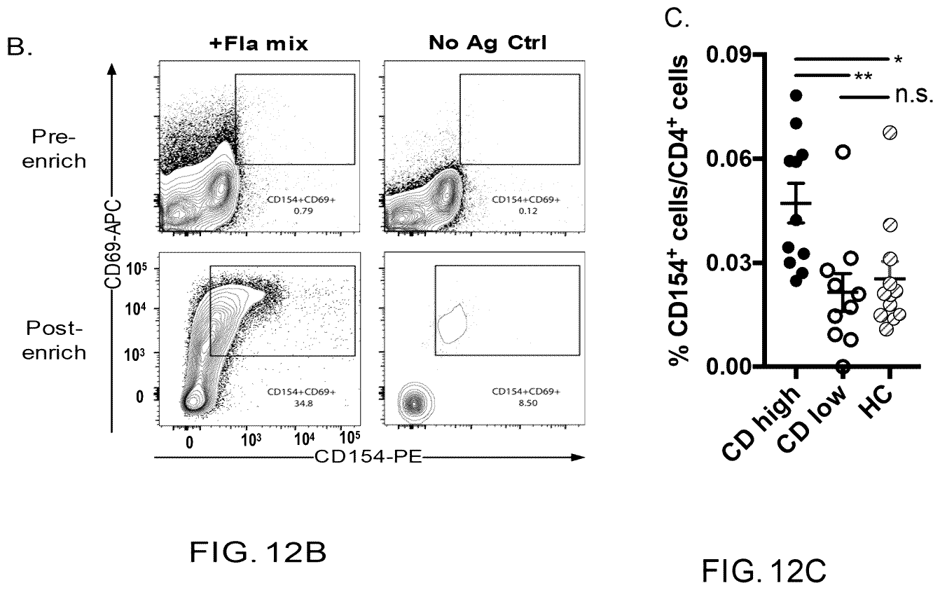

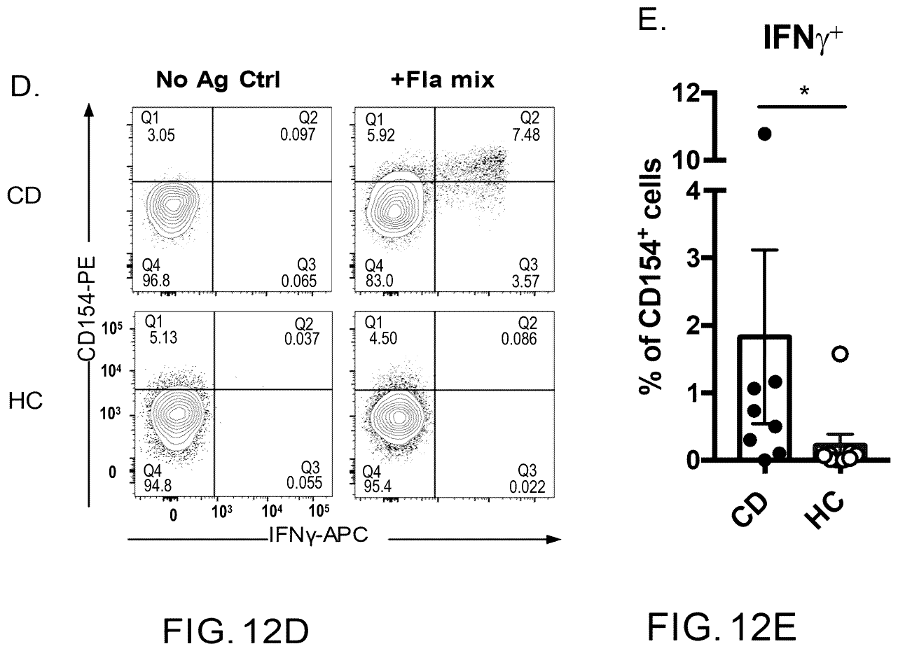

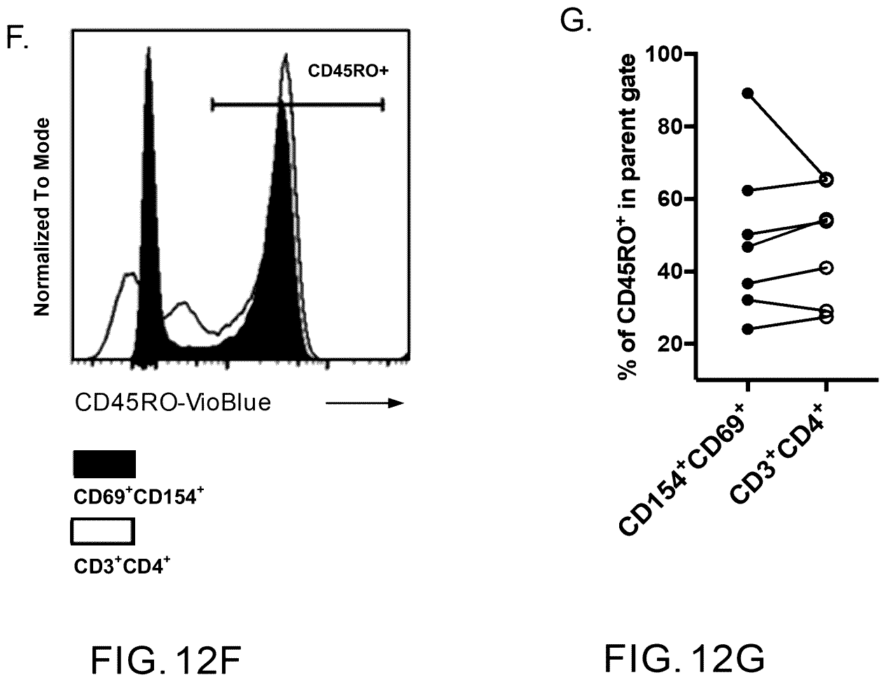

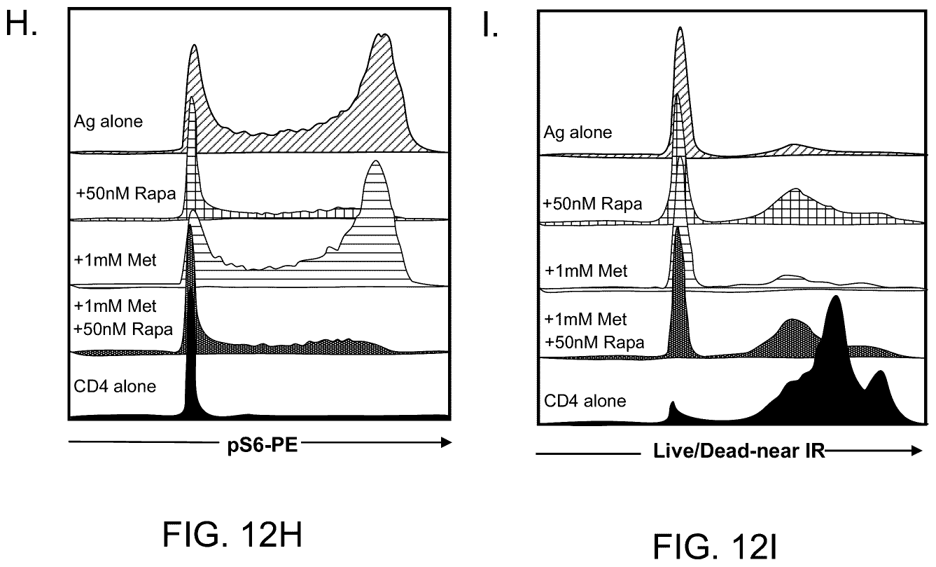

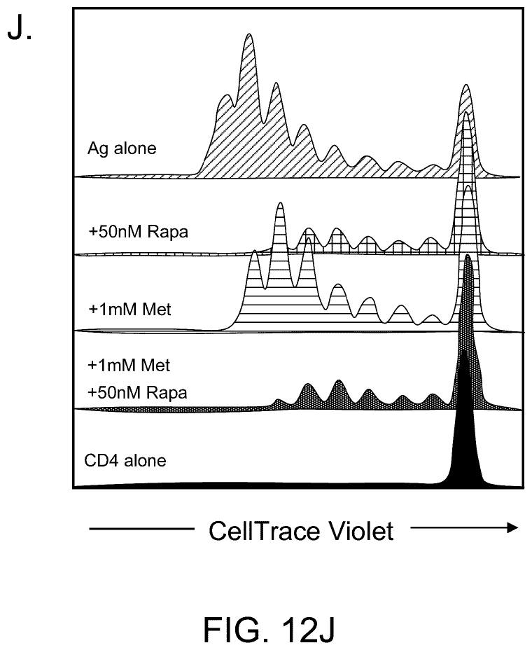

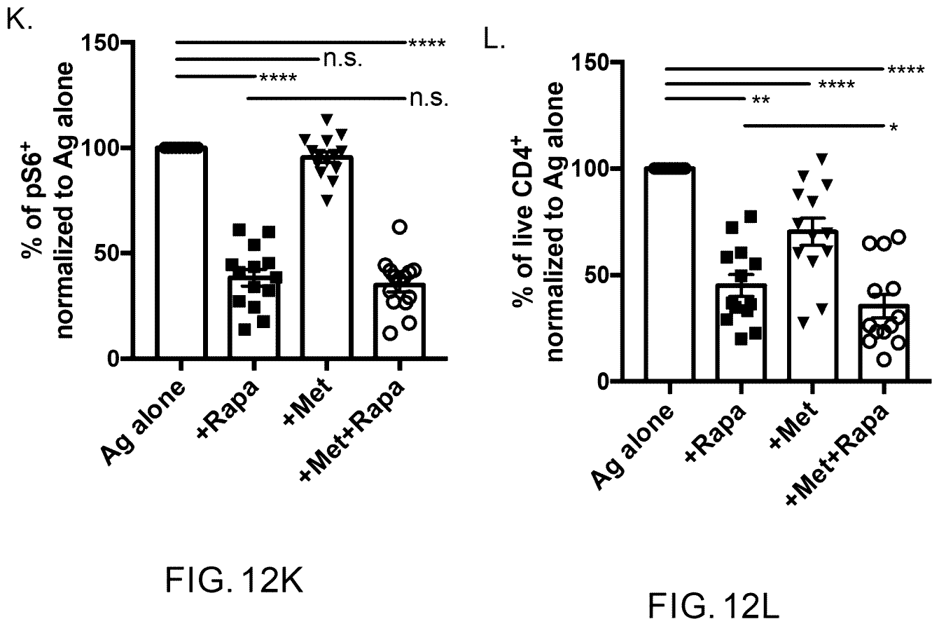

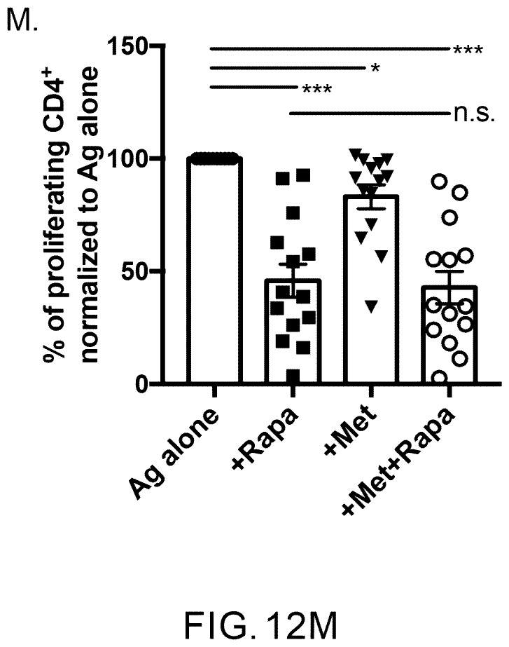

Microbiota antigen-specific CD4⁺ T_{M} cells are found circulating in both healthy people and patients with Crohn's disease. However, significantly elevated serum anti-microbiota flagellin IgG response was only found in Crohn's patients, not in healthy people, indicating a functional difference of these CD4⁺ T cells under disease conditions. Whether microbiota-specific CD4⁺ T_{M} cells in patients with Crohn's disease could be depleted with the MIdCA strategy described herein was investigated. Using the published antigen-reactive T cell enrichment (ARTE) protocol, CD154⁺CD69⁺ antigen-specific CD4⁺ T cells were isolated from peripheral blood mononuclear cells (PBMCs) obtained from patients with Crohn's disease and healthy controls upon stimulation with pooled flagellin antigens including CBir1, 14-2 Fla1, A4 Fla3, and A4 FlaX (FIG. 11A and 11B). Consistent with serologic data the frequencies of microbiota flagellin-specific CD4⁺ T cells in PBMCs were significantly elevated in patients with high serum IgG antibody response against multi-flagellin-antigens (reactive to >50% of Lachnospiraceae flagellin antigens tested), but not in patients with low anti-flagellin serum IgG or healthy donors (FIG. 11C). Also, pooled flagellin-specific CD154+ cells isolated from Crohn's patients had significantly higher expression of effector cytokines including IFNγ, TNFα, and IL17− A post 7 hr stimulation. With magnetic beads labeling, these cells were enriched from ˜0.5% to ˜30% out of the total CD4+ population (FIG. 11B) and further sorted with flow cytometry for ex vivo expansion with IL-2 and IL-7. Different from a previous study, the percentage of memory cells (CD45RO+) in flagellin-specific CD4+ T cells freshly post sorting ranged widely, from less than 20% to over 80% in different patients (FIG. 11E and 11F). Therefore, in order to target the metabolism of both naïve and memory CD4+ subsets, rapamycin, metformin, or both were tested, for their inhibitory effects on these cells post expansion. Similar to the murine data, rapamycin treatment alone upon re-stimulation with pooled flagellin greatly inhibited the phosphorylation of S6K to ˜50% reduction compared to antigen alone (FIG. 11G and 11J), leading to significantly increased cell death (FIG. 11H and 11K) and decreased cell proliferation (FIGS. 11I and 11L). As expected, metformin treatment alone had no inhibitory effect on the mTOR pathway and resulted in a lesser extent of increased cell death and decreased proliferation. Although a synergetic inhibitory effect of rapamycin and metformin on cell proliferation was not observed, the survival of flagellin-specific CD4+ T cells post re-stimulation was significantly lower than either of the drugs when applied alone. Of note, neither rapamycin or metformin treatment altered the cytokine production, such as IFNγ, TNFα and IL-17A, in microbiota flagellin-specific CD4+ T cells upon re-stimulation (FIG. 12).

### Metabolic Checkpoint Inhibition Dampens Antigen-Specific CD4⁺ T Cell Recall Response Post Ablation

In order to further determine the cellular response of remaining CBir1 TCR Tg CD4⁺ T cells post treatment when they re-encounter their cognate antigen, recipient mice with the experimental setup above were challenged with MEP1 on day 56 (FIG. 11A). The total numbers of donor CBir1Tg CD4⁺ T cells a week post challenge in mice treated with MEP1 plus different metabolic inhibitors were significantly decreased than those in mice without treatment (FIG. 11C). Further analysis showed that the phenotype of donor CBir1Tg CD4⁺ T cells was overall similar among different treatment groups, with significantly increased naïve CD4⁺ T cell population and decreased effector CD4⁺ T cells and memory CD4⁺ T cells compared to the non-treatment group, assessed by expression of CD44, CD127, and CD62L (FIG. 11B). The decrease of the memory CD4⁺ T cell population was particularly due to the ablation of effector memory CD4⁺ T cell subset (CD44⁺CD127⁺CD62L^{lo}), but not central memory CD4⁺ T cells (CD44⁺CD127⁺CD62L^{hi}) (FIG. 11D and 11E). Consistently, CD27 expression, which indicates an enhanced cell survival, was upregulated in the remaining CBir1 TCR Tg CD4⁺ T cells in mice treated with metabolic inhibitors (FIG. 11B and 11F). These results suggest that multiple rounds of intermittent peptide plus metabolic inhibition may be needed to completely convert the remaining CD4⁺ T_{CM} into T_{EM} cells in order to fully ablate the circulating microbiota-reactive memory CD4⁺ T cells in the host. Furthermore, RNA-seq on the remaining CBir1Tg CD4⁺ T cells post challenge revealed that treatment with MEP1 plus metabolic inhibition significantly affected the expression of genes involved in cell proliferation (Cxcl16, Ctsh, Tgm1, Esm1, Havcr2, Slc11a1, Vcam1, Klrg1, Igf1, Csf1r, Aif1, Itgad, cfb, Atf3, Lst1, Cks2), apoptotic process (Snca, Ctsh, Mertk, Nr1h3, Sell, Nrp1, Npas2, Slc40a1, Siah2), pro/anti-inflammatory cytokine production (Fosb, Il18bp, Il1r1, Clec7a, Slc11a1, Clec4n, Fcgr3), cell glucose and lipid metabolism (Snca, Pla2g7, C3, Lpcat2, Igf1, Pld4, Fabp5, Plbd1, Nr1h3, Hpgd), and cell signaling and trafficking (Adgrg1, Rgs16, Rgs1, Ecel1, Pdlim4, Itgae, Ccl4) (FIG. 11G), demonstrating that MEP1 plus metabolic inhibition immunotherapy ablates circulating microbiota-reactive CD4⁺ T cells by inhibiting the survival and proliferation of antigen-specific CD4⁺ T cells as well as dampening their pro-inflammatory functions.

### Metabolic Checkpoint Inhibition Dampens the Survival and Proliferation of Circulating Microbiota Antigen-Specific CD4⁺ T Cells Isolated From Crohn's Patients

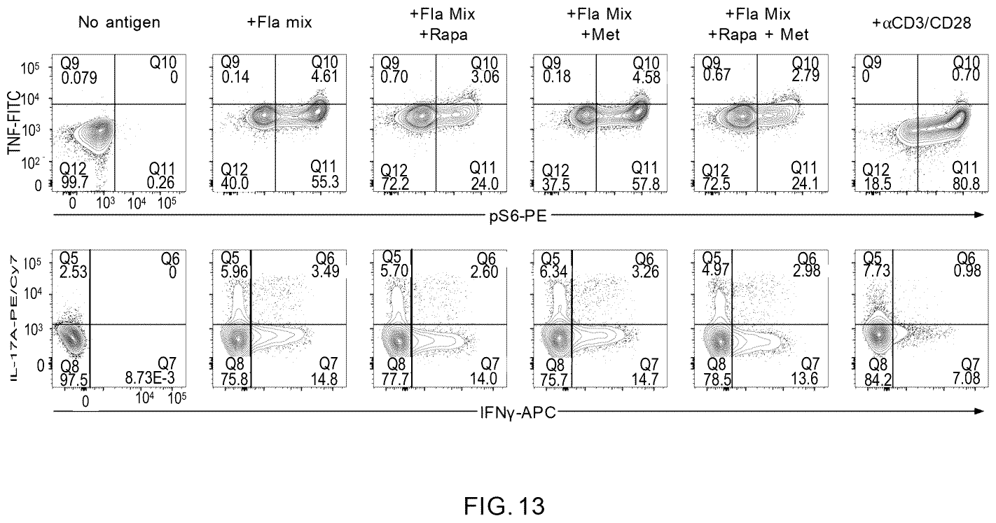

As shown in previous studies, patients with Crohn's disease have significantly elevated serum anti-microbiota flagellin IgG response compared to healthy people. Whether the circulating microbiota flagellin-specific CD4⁺ T cells are also increased in Crohn's patients was contemplated. To test this hypothesis, peripheral blood mononuclear cells (PBMCs) obtained from patients with Crohn's disease and healthy controls were stimulated with pooled flagellin antigens including CBir1, 14-2 Fla1, A4 Fla3, and FlaX. Antigen-specific CD4⁺ T cells were identified based on their up-regulation of CD154 and CD69 (FIG. 12A and 12B), an assay established from the antigen-reactive T cell enrichment (ARTE) protocol. Consistent with our serologic data (FIG. 2), the frequencies of microbiota flagellin-specific CD4⁺ T cells in PBMCs were significantly elevated in patients with high serum IgG antibody response against multiple flagellin antigens (reactive to >50% of Lachnospiraceae flagellin antigens tested), but not in patients with low anti-flagellin serum IgG or healthy donors (FIG. 12C). Also, flagellin-specific CD154⁺ cells isolated from Crohn's patients had significantly higher expression of effector cytokine IFNγ post 7 hr stimulation (FIG. 12D and 12E). With magnetic bead labeling, these cells were enriched from ˜0.5% to ˜30% out of the total CD4⁺ population (FIG. 12B) and further sort them with flow cytometry for ex vivo expansion with IL-2 and IL-7. Different from a previous study, the percentage of memory cells (CD45RO⁺) in flagellin-specific CD4⁺ T cells freshly post sorting ranged widely from less than 20% to over 80% in different patients, similar to the level of the memory subset in total CD4⁺ cells without antigen stimulation (FIG. 12F and 12G). Therefore, in order to target the metabolism of both naïve and memory CD4⁺ subsets, rapamycin, metformin, or both, were tested for their inhibitory effects on these cells post expansion. Similar to the murine data, rapamycin treatment upon re-stimulation with pooled flagellin greatly inhibited the phosphorylation of ribosomal protein S6 kinase to ˜50% reduction compared to antigen alone (FIG. 12H and 12K), leading to significantly increased cell death (FIG. 12I and 12L) and decreased cell proliferation (FIG. 12J and 12M). Metformin treatment alone had no inhibitory effect on the mTOR pathway and resulted in a lesser extent of increased cell death and decreased proliferation. Although a synergetic inhibitory effect of rapamycin and metformin on cell proliferation was not observed, the survival of flagellin-specific CD4+ T cells post re-stimulation was significantly lower than either of the drugs when applied alone. Of note, neither rapamycin or metformin treatment altered the cytokine production, such as IFNγ, TNFα and IL-17A, in microbiota flagellin-specific CD4+ T cells upon re-stimulation (FIG. 13).

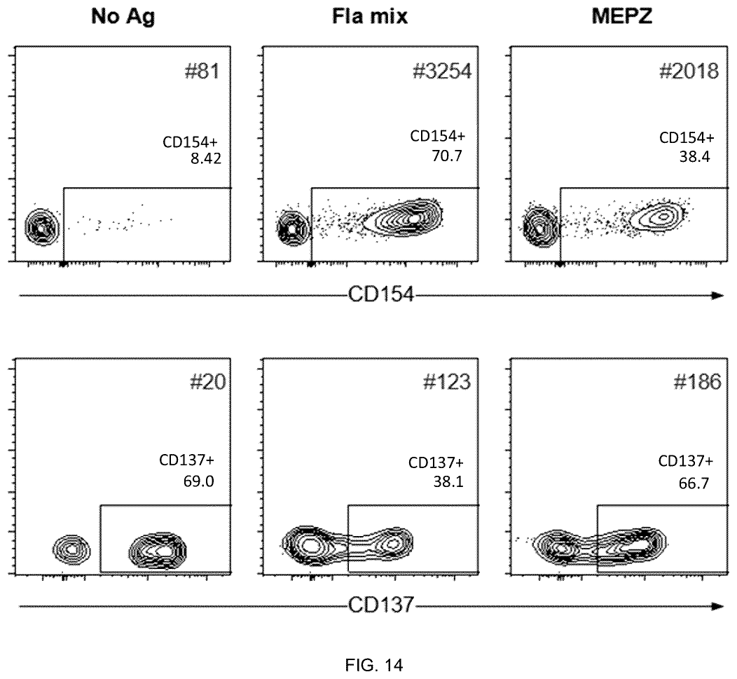

FIG. 14 shows a Crohn's patient CD4 T cell response to a flagellin multiepitope peptide (MEP1) compared to a mixture of the recombinant flagellin proteins. The multiepitope peptide (MEP1) stimulated both Tconv (CD154+) and Treg (CD137+) CD4+ T cells that are reactive to microbiota flagellin. Numbers in the upper right hand corner of each panel show absolute numbers of cells that are reactive to different stimulations.

### Preventive Immunotherapy

In some examples, the methods provided herein can be used to prevent inflammatory bowel disease, for example, Crohn's disease. For example, flagellin peptide(s) (for example, the multi-epitope peptide described herein) can be administered to a patient in remission, for example, surgery or medically induced remission. The peptide(s) can be administered to the patient intradermally or subcutaneously and an mTOR inhibitor, for example, rapamycin can be administered orally for 5 days. This administration schedule can be repeated, for example, at 2, 4, 6 or 12 month intervals.

In summary, these studies show showed that metabolic inhibition during cell activation (MIdCA) by targeting key metabolic regulators mTORC and AMPK resulted in CD4⁺ naïve and memory T cell death and anergy, but enhanced the induction of CD4⁺ regulatory T (Treg) cells. This metabolic inhibition treatment successfully prevented the development of intestinal inflammation in the CBir1 TCR Tg CD4⁺ T cell transfer colitis model. Microbiota-specific CD4⁺ T cells, especially the pathogenic T_{E} subsets, were decreased 10 fold in the intestinal lamina propria. Furthermore, the MIdCA strategy was able to prevent antigen-specific T_{M} cell formation upon initial antigen encounter, and ablate existing T_{M} cells upon re-activation in mice. Human microbiota flagellin-specific CD4⁺ T cells isolated from Crohn's patients, treated with MIdCA, were ablated in a similar manner with half of the antigen-specific T cells undergoing apoptosis. These results indicate that metabolic inhibition of activated microbiota-specific CD4⁺ T cells is an effective way to eliminate pathogenic CD4⁺ T_{M} cells and to induce Treg cells that provide antigen-specific and bystander suppression, serving as an immunotherapy for inflammatory bowel disease.

1-31. (canceled)

32. A polypeptide comprising two or more flagellin T-cell receptor (TCR) epitopes selected from the group consisting of SEQ ID NO: 1, SEQ ID NO: 2, SEQ ID NO: 3, SEQ ID NO: 6, SEQ ID NO: 7 SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO; 27, SEQ ID NO: 34, SEQ ID NO: 35, SEQ ID NO: 36, and SEQ ID NO: 37.

33. The polypeptide of claim 32, wherein the polypeptide comprises two or more T-cell receptors (TCR) epitopes selected from the group consisting of SEQ ID NO: 6 (MVVQHNMQAMNANRMLNVTT), SEQ ID NO: 7 (LTEVHSMLQRMNELAVQASNG), SEQ ID NO: 8 (MVVQHNMTAANANRM), and/or SEQ ID NO: 9 (GETHSILQRMNELATQAAN).

34. The polypeptide of claim 32, wherein the polypeptide comprises an amino acid sequence having at least 80% identity to SEQ ID NO: 5, and wherein the polypeptide is not a full-length flagellin polypeptide sequence.

35. The polypeptide of claim 32, wherein the polypeptide comprises an amino acid sequence having at least 90% identity to SEQ ID NO: 5, and wherein the polypeptide is not a full-length flagellin polypeptide sequence .

36. The polypeptide of claim 32, wherein the polypeptide comprises SEQ ID NO: 5,and wherein the polypeptide is not a full-length flagellin polypeptide sequence.

37. A pharmaceutical composition comprising a therapeutically effective amount of the polypeptide of claim 32 and a pharmaceutically acceptable carrier.

38. The pharmaceutical composition of claim 37, wherein the pharmaceutical composition is formulated for parenteral or oral administration.

39. A nucleic acid encoding the polypeptide of claim 32.

40. A vector comprising the nucleic acid of claim 39.

41. A pharmaceutical composition comprising a therapeutically effective amount of the nucleic acid of claim 39 and a pharmaceutically acceptable carrier.

42. The pharmaceutical composition of claim 41, wherein the pharmaceutical composition is formulated for parenteral or oral administration.
